SEQUENCE LISTING (48 sequences)

SEQ ID NO: 1 - protein, 120 aa, synthetic construct
QVQLQQSGPELVRPGASVKMSCKASGYTFTTYWMHWVKQRPGQGLEWIGMNDPANSEIRLNQKFKDKATLNVDKSSNTAYMQLNRLTSEDSAVYYCARWIYGSGYALDYWGQGTSVTVSS

SEQ ID NO: 2 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 3 - protein, 111 aa, synthetic construct
DIVLTQSPASLAVSLGQRATISCRASKSVSVSGYSYMHWYQQKPGQPPKLLIYLASNLESGVPARFSGSGSGTDFTLSIHPVEEEDAASYYCQLSRELPYTFGGGTKLEIK

SEQ ID NO: 4 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 5 aa, synthetic construct
TYWMH

SEQ ID NO: 6 - protein, 17 aa, synthetic construct
MNDPANSEIRLNQKFKD

SEQ ID NO: 7 - protein, 11 aa, synthetic construct
WIYGSGYALDY

SEQ ID NO: 8 - protein, 15 aa, synthetic construct
RASKSVSVSGYSYMH

SEQ ID NO: 9 - protein, 7 aa, synthetic construct
LASNLES

SEQ ID NO: 10 - protein, 9 aa, synthetic construct
QLSRELPYT

SEQ ID NO: 11 - protein, 8 aa, synthetic construct
GYTFTTYW

SEQ ID NO: 12 - protein, 8 aa, synthetic construct
NDPANSEI

SEQ ID NO: 13 - protein, 13 aa, synthetic construct
ARWIYGSGYALDY

SEQ ID NO: 14 - protein, 10 aa, synthetic construct
KSVSVSGYSY

SEQ ID NO: 15 - protein, 7 aa, synthetic construct
GYTFTTY

SEQ ID NO: 16 - protein, 6 aa, synthetic construct
DPANSE

SEQ ID NO: 17 - protein, 122 aa, synthetic construct
QVQLQQPGSELVRPGASVKLSCKASGYTFTNYWIHWVKQRHGQGLEWIGSIYPGSGIIKYDEKFKSKGTLTVDTSSNTAYMYLSSLTSEDSAVYYCTRNADGYYGDYAMDYWGQGTSVTVSS

SEQ ID NO: 18 - protein, 145 aa, synthetic construct
EFAATMHSSALLCCLVLLTGVRAQVQLQQPGSELVRPGASVKLSCKASGYTFTNYWIHWVKQRHGQGLEWIGSIYPGSGIIKYDEKFKSKGTLTVDTSSNTAYMYLSSLTSEDSAVYYCTRNADGYYGDYAMDYWGQGTSVTVSS

SEQ ID NO: 19 - protein, 107 aa, synthetic construct
DIVLTQSPAFMSASPGEKVTIICSASSSVSYMHWFQQKPGTSPRLWIYTTSNLASGVPARFSGSGSGTSYSLTISRMEAEDAATYYCQQRNSYPFTFGSGTKLEIKR

SEQ ID NO: 20 - protein, 5 aa, synthetic construct
NYWIH

SEQ ID NO: 21 - protein, 17 aa, synthetic construct
SIYPGSGIIKYDEKFKS

SEQ ID NO: 22 - protein, 13 aa, synthetic construct
NADGYYGDYAMDY

SEQ ID NO: 23 - protein, 10 aa, synthetic construct
SASSSVSYMH

SEQ ID NO: 24 - protein, 7 aa, synthetic construct
TTSNLAS

SEQ ID NO: 25 - protein, 9 aa, synthetic construct
QQRNSYPFT

SEQ ID NO: 26 - protein, 19 aa, synthetic construct
MGWSCIILFLAATATGVHS

SEQ ID NO: 27 - protein, 20 aa, synthetic construct
METDTLLLWVLLLWVPGSTG

SEQ ID NO: 28 - DNA, 360 bp, synthetic construct
  source 1..360: mol_type = other DNA
caggtccaactgcagcaatctgggcctgagctggtgaggcctggggcttcagtgaagatgtcctgcaaggcttcaggctataccttcaccacctactggatgcactgggtgaaacagaggcctggacaaggccttgagtggattggcatgaatgatcctgcgaatagtgaaattaggttaaatcagaaattcaaggacaaggccacattgaatgtagacaaatcctccaacacagcctacatgcagctcaacagactgacatctgaggactctgcagtctattactgtgcaagatggatctacggtagtggctacgctttggactactggggtcaaggaacctcagtcaccgtctcctca

SEQ ID NO: 29 - DNA, 972 bp, synthetic construct
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 30 - DNA, 333 bp, synthetic construct
  source 1..333: mol_type = other DNA
gacattgtgctgacacagtctcctgcttccttagctgtatctctggggcagagggccaccatctcatgcagggccagcaaaagtgtcagtgtatctggctatagttatatgcactggtaccaacagaaaccaggacagccacccaaactcctcatctatcttgcatccaacctagaatctggggtccctgccaggttcagtggcagtgggtctgggacagacttcaccctcagcatccatcctgtggaggaggaggatgctgcatcctattactgtcagctcagtagggagcttccgtacacgttcggcggggggaccaagctggaaataaaa

SEQ ID NO: 31 - DNA, 321 bp, synthetic construct
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 32 - DNA, 366 bp, synthetic construct
  source 1..366: mol_type = other DNA
caggtccaactacagcaacctgggtctgagctggtgaggcctggagcttcagtgaagctgtcctgcaaggcttctggctacacattcaccaactactggatacactgggtgaagcagagacatggacaaggccttgagtggattggaagtatttatcctggtagtggtataattaaatacgatgaaaaattcaagagtaagggtacattgactgtagacacatcctccaacacagcctacatgtacctcagcagcctgacatctgaggactctgcggtctattactgtacaagaaacgccgatggttactatggtgactatgctatggactactggggtcaaggaacctcagtcaccgtctcctca

SEQ ID NO: 33 - DNA, 435 bp, synthetic construct
  source 1..435: mol_type = other DNA
gaattcgccgccaccatgcacagctcagcactgctctgttgcctggtcctcctgactggggtgagggcccaggtgcagctgcagcagcccggcagcgagctggtgaggcctggagcttccgtgaagctgtcctgcaaggcctccggctacacattcaccaactactggattcactgggtgaagcagaggcacggccagggcctggagtggatcggctccatctaccccggcagcggcatcatcaagtacgatgagaagtttaagagcaagggcaccctgaccgtggatacaagcagcaacacagcctacatgtacctgagctccctgacaagcgaggacagcgccgtgtactactgcaccagaaatgccgatggctactacggcgactacgccatggactactggggccagggcaccagcgtgacagtgtcctcc

SEQ ID NO: 34 - DNA, 321 bp, synthetic construct
  source 1..321: mol_type = other DNA
gacattgtgctgacacaatctccagcattcatgtctgcatctccaggggagaaggtcaccataatctgcagtgccagctcaagtgtaagttacatgcactggttccagcagaagccaggcacttctcccagactctggatttataccacatccaacctggcttctggagtccctgctcgcttcagtggcagtggatctgggacctcttactctctcacaatcagccgaatggaggctgaagatgctgccacttattactgccagcaaaggaatagttacccattcacgttcggctcggggacaaagttggaaataaaacgg

SEQ ID NO: 35 - DNA, 57 bp, synthetic construct
  source 1..57: mol_type = other DNA
atgggatggagctgtatcatcctcttcttggcagcaacagctacaggtgtccactcc

SEQ ID NO: 36 - DNA, 60 bp, synthetic construct
  source 1..60: mol_type = other DNA
atggagacagacacactcctgttatgggtactgctgctctgggttccaggttccactggt

SEQ ID NO: 37 - protein, 243 aa, Homo sapiens
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 38 - protein, 171 aa, Mus musculus
MHPQGRAAPPQLLLGLFLVLLLLLQLSAPISASENPKVKQKALIRQREVVDLECTFTKMRSNSALRVLFSGSLRLKCRNACCQRWYFTFNGAECSGPLPIEAIIYLDQGSPELNSTINIHRTSSVEGLCEGIGAGLVDVAIWVGTCSDYPKGDASTGWNSVSRIIIEELPK

SEQ ID NO: 39 - protein, 15 aa, synthetic construct
TRNADGYYGDYAMDY

SEQ ID NO: 40 - protein, 5 aa, synthetic construct
SSVSY

SEQ ID NO: 41 - protein, 7 aa, synthetic construct
GYTFTNY

SEQ ID NO: 42 - protein, 6 aa, synthetic construct
YPGSGI

SEQ ID NO: 43 - protein, 129 aa, synthetic construct
EFAATMHSSALLCCLVLLTGVRADIVLTQSPAFMSASPGEKVTIICSASSSVSYMHWFQQKPGTSPRLWIYTTSNLASGVPARFSGSGSGTSYSLTISRMEAEDAATYYCQQRNSYPFTFGSGTKLEIK

SEQ ID NO: 44 - protein, 8 aa, synthetic construct
GYTFTNYW

SEQ ID NO: 45 - protein, 8 aa, synthetic construct
IYPGSGII

SEQ ID NO: 46 - DNA, 387 bp, synthetic construct
  source 1..387: mol_type = other DNA
gaattcgccgccaccatgcacagctcagcactgctctgttgcctggtcctcctgactggggtgagggccgacatcgtgctgacacagagccctgcctttatgtccgccagccctggcgagaaggtgaccatcatctgctccgcctccagcagcgtgagctacatgcactggtttcagcagaagcccggcaccagccctagactgtggatctacaccacctccaacctggcctccggcgtgcccgctaggttctccggaagcggctccggcacaagctacagcctgaccatcagcaggatggaggccgaggacgccgccacatactactgtcagcagagaaattcctacccttttacctttggctccggcaccaagctggagatcaag

SEQ ID NO: 47 - protein, 47 aa, synthetic construct
AKTTPPSVYPLAPGSAAQTNSMVTLGCLVKGYFPEPVTVTWNSGSLS

SEQ ID NO: 48 - DNA, 141 bp, synthetic construct
  source 1..141: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtcc

(19) United States
(12) Patent Application Publication
(10) Pub. No.: US 2026/0265355 A1
(43) Pub. Date: Sep. 10, 2026

(54) CTHRC1 IS A NOVEL IMMUNE CHECKPOINT MOLECULE IN COLORECTAL CANCER

(71) Applicant: MaineHealth, Portland, ME (US)

(72) Inventors: Michaela Reagan, South Portland, ME (US); Volkhard Lindner, South Portland, ME (US); Sergey Ryzhov, Cape Elizabeth, ME (US)

(73) Assignee: MaineHealth, Portland, ME (US)

(21) Appl. No.: 19/558,859

(22) Filed: Mar. 6, 2026

Related U.S. Application Data
(60) Provisional application No. 63/768,391, filed on Mar. 7, 2025.

(51) Int. Cl. C07K 16/18; A61K 39/00; A61K 47/68; A61P 35/00

(52) U.S. Cl. CPC C07K 16/18; A61K 47/68031; A61K 47/68033; A61K 47/68035; A61K 47/6809; A61K 47/6843; A61P 35/00; A61K 2039/505; C07K 2317/52; C07K 2317/565; C07K 2317/77

(57) ABSTRACT

Disclosed herein are Collagen Triple Helix Repeat Containing 1 (CTHRC1) antibodies, including compositions and methods of using said antibodies. The antibody molecules can be used to treat, prevent, and/or diagnose disorders and diseases, such as colorectal cancer and pre-cancerous cells of colorectal cancer.

### RELATED APPLICATIONS

The instant application claims priority to U.S. Provisional Application No. 63/768,391, filed Mar. 7, 2025, the entire contents of which are expressly incorporated by reference herein.

### REFERENCE TO ELECTRONIC SEQUENCE LISTING

The application contains a Sequence Listing which has been submitted electronically in XML format and is hereby incorporated by reference in its entirety. Said .XML copy, created on Mar. 5, 2026, is named “136191-00520.xml” and is 47,664 bytes in size. The sequence listing contained in this .XML file is part of the specification and is hereby incorporated by reference herein in its entirety.

### BACKGROUND OF THE INVENTION

There are currently over one million people in the US fighting colorectal cancer (CRC), and from 2000-2021, nearly 1.2 million people died of this disease. Moreover, there is an overall lifetime risk of developing CRC of about 1 in 23 for men and 1 in 25 for women in the USA. Although CRC is frequently, but not always, fatal, it can also have detrimental long-term physical and emotional effects on an individual's life due to both the disease and side effects of currently available therapies. The rates of CRC in northern New England (Vermont, New Hampshire, and Maine) are similar to national averages, demonstrating an urgent unmet need for more effective, less toxic therapies for our local community members, as well as patients across the world.

Immunosuppression in the tumor microenvironment is a major obstacle to prevention and successful treatment of CRC and tumor-associated macrophages (TAMs) are some of the major factors responsible for immune suppression in CRC, as they accelerate tumor growth by inhibiting tumor-killing immune cells.

Twenty years ago, a novel protein, Collagen Triple Helix Repeat Containing 1 (CTHRC1), was discovered as a secreted protein in injured and diseased arteries that inhibits collagen expression and promotes cell migration [1]. Ten years later, CTHRC1/CTHRC1 was found to be expressed by activated stroma in human colorectal cancer (CRC), and other cancers (gastric, breast, endometrial, pancreatic, kidney, lung and skin), but not typically expressed by tumor cells themselves [2]. There is still a need for the development of antibodies for diagnosing and treating various types of cancer and preventing cancers from developing, such as antibodies targeting CTHRC1.

### SUMMARY OF THE INVENTION

This disclosure provides, at least in part isolated antibody, or antigen-binding fragment thereof that bind to Collagen Triple Helix Repeat Containing 1 (CTHRC1), e.g., human CTHRC1 (e.g., human CTHRC1 comprising the amino acid sequence of SEQ ID NO: 37), and that comprise one or more functional and structural properties disclosed herein. In an embodiment, the isolated antibody, or antigen-binding fragment thereof binds to and/or reduces (e.g., inhibits, blocks, or neutralizes) one or more activities of CTHRC1.

Accordingly, in one aspect, the present invention provides an isolated antibody, or antigen-binding fragment thereof, which can include a heavy (H) chain variable domain sequence (abbreviated herein as VH), and a light (L) chain variable domain sequence (abbreviated herein as VL). The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

In one aspect, the present invention provides an isolated antibody, or antigen-binding fragment thereof, which comprises a) a heavy chain variable region (VH) comprising an HCDR1 amino acid sequence of SEQ ID NO: 5; an HCDR2 amino acid sequence of SEQ ID NO: 6; and an HCDR3 amino acid sequence of SEQ ID NO: 7; and a light chain variable region (VL) comprising an LCDR1 amino acid sequence of SEQ ID NO: 8, an LCDR2 amino acid sequence of SEQ ID NO: 9, and an LCDR3 amino acid sequence of SEQ ID NO: 10; or b) VH comprising an HCDR1 amino acid sequence of SEQ ID NO: 20; an HCDR2 amino acid sequence of SEQ ID NO: 21; and an HCDR3 amino acid sequence of SEQ ID NO: 22; and VL comprising an LCDR1 amino acid sequence of SEQ ID NO: 23, an LCDR2 amino acid sequence of SEQ ID NO: 24, and an LCDR3 amino acid sequence of SEQ ID NO: 25, wherein the HCDR1, HCDR2, HCDR3, LCDR1, LCDR2, and LCDR3 are according to Kabat numbering.

In another aspect, the present invention provides an isolated antibody, or antigen-binding fragment thereof, which comprises a) a heavy chain variable region (VH) comprising an HCDR1 amino acid sequence of SEQ ID NO: 11; an HCDR2 amino acid sequence of SEQ ID NO: 12; and an HCDR3 amino acid sequence of SEQ ID NO: 13; and a light chain variable region (VL) comprising an LCDR1 amino acid sequence of SEQ ID NO: 14, an LCDR2 amino acid sequence of “LAS”, and an LCDR3 amino acid sequence of SEQ ID NO: 10; or b) VH comprising an HCDR1 amino acid sequence of SEQ ID NO: 37; an HCDR2 amino acid sequence of SEQ ID NO: 38; and an HCDR3 amino acid sequence of SEQ ID NO: 39; and VL comprising an LCDR1 amino acid sequence of SEQ ID NO: 40, an LCDR2 amino acid sequence of “TT”, and an LCDR3 amino acid sequence of SEQ ID NO: 25, wherein the HCDR1, HCDR2, HCDR3, LCDR1, LCDR2, and LCDR3 are according to IMGT numbering.

In another aspect, the present invention provides an isolated antibody, or antigen-binding fragment thereof, which comprises a) a heavy chain variable region (VH) comprising an HCDR1 amino acid sequence of SEQ ID NO: 15; an HCDR2 amino acid sequence of SEQ ID NO: 16; and an HCDR3 amino acid sequence of SEQ ID NO: 7; and a light chain variable region (VL) comprising an LCDR1 amino acid sequence of SEQ ID NO: 8, an LCDR2 amino acid sequence of SEQ ID NO: 9, and an LCDR3 amino acid sequence of SEQ ID NO: 10; or b) VH comprising an HCDR1 amino acid sequence of SEQ ID NO: 41; an HCDR2 amino acid sequence of SEQ ID NO: 42; and an HCDR3 amino acid sequence of SEQ ID NO: 22; and VL comprising an LCDR1 amino acid sequence of SEQ ID NO: 23, an LCDR2 amino acid sequence of SEQ ID NO: 24, and an LCDR3 amino acid sequence of SEQ ID NO: 25, wherein the HCDR1, HCDR2, HCDR3, LCDR1, LCDR2, and LCDR3 are according to Chothia numbering.

In one embodiment, the VH comprises an amino acid sequence at least 85%, 90%, or 95% identity to SEQ ID NO: 1 or 17. In one embodiment, the VH comprises an amino acid sequence set forth in SEQ ID NO: 1 or 17. In one embodiment, the VH consists of an amino acid sequence set forth in SEQ ID NO: 1 or 17.

In one embodiment, the VL comprises an amino acid sequence at least 85%, 90%, or 95% identity to SEQ ID NO: 3 or 19. In one embodiment, the VL comprises an amino acid sequence set forth in SEQ ID NO: 3 or 19. In one embodiment, the VL consists of an amino acid sequence set forth in SEQ ID NO: 3 or 19.

In one embodiment, the VH comprises an amino acid sequence at least 85%, 90%, or 95% identity to any of SEQ ID NOs: 1 and 17, and the VL comprises an amino acid sequence at least 85%, 90%, or 95% identity to SEQ ID NO: 3 or 19. In one embodiment, the VH comprises an amino acid sequence set forth in SEQ ID NO: 1 or 17, and the VL comprises an amino acid sequence set forth in SEQ ID NO: 3 or 19.

In one embodiment, the VH consists of an amino acid sequence set forth in SEQ ID NO: 1 or 17 and the VL consists of an amino acid sequence set forth in SEQ ID NO: 3 or 19. In one embodiment, the VH comprises an amino acid sequence set forth in SEQ ID NO: 1 and the VL comprises an amino acid sequence set forth in SEQ ID NO: 3. In one embodiment, the VH comprises an amino acid sequence set forth in SEQ ID NO: 17 and the VL comprises an amino acid sequence set forth in SEQ ID NO: 19.

In one embodiment, the isolated antibody, or antigen-binding fragment thereof comprises an antigen-binding fragment. In one embodiment, the antigen-binding fragment comprises a Fab, F(ab′)2, Fv, dsFv, sc(Fv)2, scFv, a diabody, affibody, avimer, or nanobody.

In one embodiment, the heavy chain constant region is chosen from the heavy chain constant regions of IgG1, IgG2, IgG3, or IgG4, or a chimera of two or more isotypes (e.g. IgG2/4). In one embodiment, the light chain constant region is chosen from the light chain constant regions of kappa or lambda. In one embodiment, the heavy chain constant region is chosen from the heavy chain constant regions of IgG1, IgG2, IgG3, IgG4, or a chimera of two or more isotypes (e.g. IgG2 and IgG4), and the light chain constant region is chosen from the light chain constant regions of kappa or lambda.

In one embodiment, the isolated antibody, or antigen-binding fragment thereof comprises an Fc region.

In one embodiment, the isolated antibody, or antigen-binding fragment thereof comprises two VH and two VL.

In one embodiment, the isolated antibody, or antigen-binding fragment thereof is a humanized antibody, monoclonal antibody, synthetic antibody, monospecific antibody, multispecific antibody, and/or bispecific antibody.

In one embodiment, the said isolated antibody, or antigen-binding fragment thereof specifically binds to Collagen Triple Helix Repeat Containing 1 (CTHRC1) or a fragment thereof. In one embodiment, the CTHRC1 is a human CTHRC1 or a fragment thereof.

In another aspect, the present invention provides an isolated antibody, or antigen-binding fragment thereof that competes for binding to CTHRC1 or a fragment thereof with an isolated antibody, or antigen-binding fragment thereof as disclosed herein.

In another aspect, the present invention provides an isolated antibody, or antigen-binding fragment thereof that binds to the same or overlapping epitope as the epitope recognized by an isolated antibody, or antigen-binding fragment thereof as disclosed herein.

In another aspect, the present invention provides a pharmaceutical composition comprising the isolated antibody, or antigen-binding fragment thereof as disclosed herein, and a pharmaceutically acceptable carrier, excipient or stabilizer.

In another aspect, the present invention provides an isolated nucleic acid encoding the VH and VL of the isolated antibody, or antigen-binding fragment thereof as disclosed herein.

In another aspect, the present invention provides an expression vector comprising the nucleic acid as disclosed herein.

In another aspect, the present invention provides a host cell comprising the nucleic acid as disclosed herein or the vector as disclosed herein.

In another aspect, the present invention provides a method of producing an isolated antibody, or antigen-binding fragment thereof, comprising culturing the host cell as disclosed herein under conditions suitable for gene expression.

In another aspect, the present invention provides a method of binding or interacting with CTHRC1 or a fragment thereof, comprising contacting CTHRC1 with an isolated antibody, or antigen-binding fragment thereof as disclosed herein, or a pharmaceutical composition as disclosed herein.

In another aspect, the present invention provides a method of treating a disease or disorder, comprising administering to a subject in need thereof an isolated antibody, or antigen-binding fragment thereof as disclosed herein, or a pharmaceutical composition as disclosed herein, in an amount effective to treat the disease.

In one embodiment, the disorder or disease is a cancer, optionally, wherein the cancer is chosen from the group comprising of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer. In one embodiment, the disorder or disease is a cancer, optionally, wherein the cancer is colorectal cancer.

In one embodiment, the disease or disorder is pre-cancerous cells in the subject, optionally wherein the pre-cancerous cells are associated with one or more of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer. In one embodiment, the pre-cancerous cells are associated with colorectal cancer.

In one embodiment, the method further comprises administering a second therapeutic agent or modality.

In one embodiment, the second therapeutic agent or modality is administered before, during, or after the isolated antibody, or antigen-binding fragment thereof is administered.

In another aspect, the present invention provides a method of preventing a disease or disorder, comprising administering to a subject in need thereof an isolated antibody, or antigen-binding fragment thereof as disclosed herein, or a pharmaceutical composition as disclosed herein, in an amount effective to prevent the disorder or disease.

In one embodiment, the disease is a cancer, optionally, wherein the cancer is chosen from the group comprising of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer.

In one embodiment, the disease or disorder is pre-cancerous cells in the subject, optionally wherein the pre-cancerous cells are associated with one or more of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer.

In another aspect, the present invention provides a method of identifying a subject that would benefit, comprising (i) contacting a sample or a subject with an isolated antibody, or antigen-binding fragment thereof as disclosed herein under conditions that allow interaction of the isolated antibody, or antigen-binding fragment thereof and CTHRC1 to occur, and (ii) detecting formation of a complex between the isolated antibody, or antigen-binding fragment thereof and the sample or subject.

In another aspect, the present invention provides an antibody-drug conjugate (ADC) comprising the antibody, or antigen-binding fragment thereof as disclosed herein conjugated to at least one drug, wherein the antibody, or antigen-binding fragment thereof binds collagen triple helix repeat containing 1 (CTHRC1).

In one embodiment, the at least one drug is selected from the group consisting of an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a DNA damaging agent, a corticosteroid, a photoactive therapeutic agent, an oligonucleotide, a radionuclide agent, a radiosensitizer, a topoisomerase inhibitor, and a tyrosine kinase inhibitor.

In one embodiment, the mitotic inhibitor is selected from the group consisting of monomethyl auristatin E (MMAE), monomethyl auristatin F (MMAF), mertansine (DM1), and ravtansine (DM4).

In one embodiment, the DNA damaging agent is selected from the group consisting of psilocybin, gliclazomycin, streptomycin, pyrrolobenzodiazepine (PBD), doxorubicin, and adrianmycin.

In one embodiment, the at least one drug is conjugated to the antibody, or antigen-binding portion thereof, via a linker. In one embodiment, the linker is cleavable or a non-cleavable linker.

In another aspect, the present invention provides a pharmaceutical composition comprising the ADC as disclosed herein, and a pharmaceutically acceptable carrier.

In another aspect, the present invention provides a method of treating or preventing a disease or disorder, comprising administering to a subject a therapeutically effective amount of the ADC as disclosed herein, or the pharmaceutical composition as disclosed herein, thereby treating or preventing the disease or disorder.

In one embodiment, the disease is a cancer, optionally, wherein the cancer is chosen from the group comprising of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer.

In one embodiment, the disease or disorder is pre-cancerous cells in the subject, optionally wherein the pre-cancerous cells are associated with one or more of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIGS. 1A-1E show Cthrc1 null (KO) and WT mice in pre-determined endpoint study. FIG. 1A shows BLI imaging of whole mouse tumor burden in WT mice and Cthrc1 KO mice over time, photons per second (p/s), n=10. Student's T-tests at day 13 and 14. FIG. 1B shows representative WT (top) and Cthrc1 KO (bottom) BLI images at day 16. FIG. 1C shows H&E immunohistochemistry of tumors from Cthrc1 KO (right) and WT (left) mice. FIG. 1D shows flow cytometry results of CD3+ T cells in tumors of Cthrc1 KO and WT mice, day 16. Mann-Whitney test. FIG. 1E shows flow cytometry results of CD3+ T cells in spleens of Cthrc1 KO and WT mice, day 16. Mann-Whitney test. Data represent mean±SEM; individual mice shown with data points.

FIGS. 2A-2D show Cthrc1 null (KO) and WT mice in survival study. FIG. 2A shows BLI imaging of whole mouse tumor burden in WT mice and Cthrc1 KO mice over time, photons per second (p/s), n=10. P value from two-way ANOVA analysis from FIG. 2A. FIG. 2B shows representative WT (top) and Cthrc1 KO (bottom) BLI images over time. FIG. 2C shows in vivo caliper measurements of each tumor. FIG. 2D shows Kaplan-Meier survival curve of individual mice.

FIGS. 3A-3B show WT mice treated with CTHRC1 Abs. FIG. 3A shows BLI imaging of whole mouse tumor burden of anti-CTHRC1 antibody-treated mice over 18 days, with 4 total treatments (Flux, photons per second, p/s), n=11. Data are individual mice. FIG. 3B shows BLI overlay of data from FIG. 3A with data from our first Cthrc1 KO study. Data are group Mean±SEM.

FIGS. 4A-4C show immunosuppressive effect of CTHRC1 on granulocyte-macrophage colony-stimulating factor (GM-CSF), commonly found within the tumor setting, and by macrophage-CSF (M-CSF): recombinant CTHRC1 promotes GM-CSF-induced differentiation of myeloid cells. FIG. 4A shows representative flow cytometric plots which show the effects of 20 ng/ml M-CSF (upper panel) and 20 ng/ml GM-CSF (lower) on the differentiation of BM Lin-negative hematopoietic progenitor cells (HPCs) from immature myeloid cells (Ly6C+ only) toward terminally differentiated mononuclear cells (MHCII+, F4/80+, and/or CD11c+, and Ly6C−). FIG. 4B shows percentage of transitioning cells (positive for all markers) during differentiation with M-CSF (circles) and GM-CSF (squares), n=5; P values for M-CSF (lower) and GM-CSF (upper) between day 3 and 5 (left) and day 5 and 7 (right) are indicated; two-way ANOVA, Sidak's post hoc test. FIG. 4C shows flow cytometric plots which shows the effect of CTHRC1 (200 ng/ml) on GM-CSF-induced differentiation of myeloid cells, representative of n=5.

FIGS. 5A-5E show comparison of tumor growth in WT mice with that in Cthrc1 null (KO) mice. FIG. 5A shows a schematic of an experimental design. At 1-week post-inoculation, tumor burden in Cthrc1tm1VliC57BL/6J (KO) mice and C57BL/6J (WT) were monitored with BLI measurement. At the endppint of Day 70, mice were euthanized and tumor weighed. FIG. 5B shows WT and Cthrc1 null (KO) mouse weight over time post-inoculation. FIG. 5C shows in vivo caliper measurements of each tumor. FIG. 5D shows BLI imaging of whole mouse tumor burden in WT and Cthrc1 KO mice over time, photons per second (p/s). FIG. 5E shows Kaplan-Meier survival curve of individual mice.

FIGS. 6A and 6B show binding of VVD-CTHRC1 (green) is detected in a population of cells (nuclei in blue) isolated from Cthrc1 null mouse spleens (FIG. 6A) by confocal microscopy. A higher magnification of the upper right portion of (FIG. 6A) is shown in (FIG. 6B) with VVD-CTHRC1 appearing in a fine punctate pattern consistent with binding to a cell surface receptor.

FIGS. 7A-7D show confocal images of CHO-K1 cell transfected with a TIMD4 (FIGS. 7A-7C) or PAQR9 (FIG. 7D) expression construct are shown. Cells were incubated with VVD-CTHRC1 (FIGS. 7A, 7B, and 7D) or control conditioned medium (FIG. 7C). Visualization of bound VVD-CTHRC1 is seen in green only in cells expressing TIMD4 seen in red (FIGS. 7A and 7B). Co-localization of bound VVD-CTHRC1 and TIMD4 is seen in yellow-orange in the merged images (FIGS. 7A and 7B). Control conditioned medium shows no binding of VVD-CTHRC1 to TIMD4 expressing cells (FIG. 7C). Cells transfected with the cell surface receptor PAQR9 and exposed to VVD-CTHRC1 show that this receptor does not bind VVD-CTHRC1 (FIG. 7D), demonstrating that binding of VVD-CTHRC1 is specific to TIMD4.

### DETAILED DESCRIPTION

Disclosed herein are antibody, or antigen-binding fragment thereof that bind to CTHRC1, e.g., human CTHRC1, with high affinity and specificity. Advantageously, several of the antibody, or antigen-binding fragment thereof describe herein have improved ability to reduce (e.g., inhibit, block, or neutralize) one or more biological activities of CTHRC1. Nucleic acid molecules encoding the antibody, or antigen-binding fragment thereof, expression vectors, host cells, compositions (e.g., pharmaceutical compositions), kits, and methods for making the antibody, or antigen-binding fragment thereof, are also provided. The antibody, or antigen-binding fragment thereof and pharmaceutical compositions disclosed herein can be used (alone or in combination with other agents or therapeutic modalities) to treat, prevent and/or diagnose disorders and diseases, e.g., disorders and diseases associated with activation of CTHRC1.

### Definitions

In order that the invention may be more readily understood, certain terms are first defined. In addition, it should be noted that whenever a value or range of values of a parameter are recited, it is intended that values and ranges intermediate to the recited values are also intended to be part of this invention.

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “or” is used herein to mean, and is used interchangeably with, the term “and/or”, unless context clearly indicates otherwise.

The term “about” and “approximately” are used herein to mean within the typical ranges of tolerances in the art. For example, “about” can be understood as about 2 standard deviations from the mean. According to certain embodiments, about means±10%. According to certain embodiments, about means±5%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

Ranges provided herein are understood to include all individual integer values and all subranges within the ranges.

As used herein, the term “in combination with,” is not intended to imply that the therapy or the therapeutic agents must be administered at the same time and/or formulated for delivery together, although these methods of delivery are within the scope described herein. The therapeutic agents can be administered concurrently with, prior to, or subsequent to, one or more other additional therapies or therapeutic agents.

The term “active agent” refers to the ingredient, component or constituent of the compositions of the described invention responsible for the intended therapeutic effect.

The term “administer” as used herein means to give or to apply. The term “administering” as used herein includes in vivo administration, as well as administration directly to tissue ex vivo. “Administering” may be accomplished by any route as disclosed below.

In the context of an amino acid sequence, the term “substantially identical” is used herein to refer to a first amino acid that contains a sufficient or minimum number of amino acid residues that are a) identical to, or b) conservative substitutions of aligned amino acid residues in a second amino acid sequence such that the first and second amino acid sequences can have a common structural domain and/or common functional activity; or in the context of a nucleic acid, the term “substantially identical” is used to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, amino acid sequences that contain a common structural domain having at least about 80%, 85%, 90%, 91%, 92%, 93%, 94%, 95%, 96%, 97%, 98% or 99% identity to a reference sequence, e.g., a sequence provided herein. The compositions and methods disclosed herein encompass polypeptides and nucleic acids having the sequences specified, or sequences substantially identical or similar thereto, e.g., sequences at least 80%, 85%, 90%, 91%, 92%, 93%, 94%, 95%, 96%, 97%, 98% or 99% identical or higher to the sequence specified. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm, such as, but not limited to, Clustal Omega (Sievers et al. Mol Syst Biol. 2011; 7:539), Kalign2 (Lassmann et al. Nucleic Acids Res. 2009; 37 (3): 858-65; Lassmann and Sonnhammer BMC Bioinformatics. 2005; 6:298), MAFFT (Katoh and Standley Mol Biol Evol. 2013; 30 (4): 772-80), MUSCLE (Edgar Nucleic Acids Res. 2004; 32 (5): 1792-7; Edgar BMC Bioinformatics. 2004; 5:113), MView (Brown et al. Bioinformatics. 1998; 14 (4): 380-1)

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

As used herein, the terms “antigen” refers to any substance that elicits an immune response.

The term “antigen-binding site” as used herein refers to the site at the tip of each arm of an antibody that makes physical contact with an antigen and binds it noncovalently. The antigen specificity of the antigen-binding site is determined by its shape and the amino acids present.

The term “antigenic determinant” or “epitope” as used herein refers to that portion of an antigenic molecule that is contacted by the antigen-binding site of a given antibody or antigen receptor.

The term “binding” and its other grammatical forms is used to mean a lasting attraction between chemical substances.

The term “binding motif” as used herein refers to sequences for specific binding. For example, sequence motifs are short, recurring patterns in DNA that are presumed to have a biological function, which often indicate sequence-specific binding sites for proteins such as nucleases and transcription factors (TF). Transcription factor binding motifs (TFBMs) are genomic sequences that specifically bind to transcription factors.

The term “binding specificity” as used herein refers to both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” e.g., a CTHRC1-associated disorder or disease and/or CTHRC1-associated cancer, means that a subject (e.g., a human) who has a disorder or disease, e.g., a CTHRC1-associated disorder or disease and/or CTHRC1-associated cancer, and/or experiences a symptom of a disorder or disease, will, in an embodiment, suffer less a severe symptom and/or recover faster when an antibody, or antigen-binding fragment thereof is administered than if the antibody, or antigen-binding fragment thereof were never administered. In an embodiment, when a CTHRC1-associated disorder or disease and/or CTHRC1-associated cancer, is treated, the level of CTHRC1 may be lower in a treated subject compared to a comparable untreated subject. For example, a diagnostic assay using immunofluorescence or electron microscopy will detect CTHRC1 in a biological sample of a subject after administration of an antibody, or antigen-binding fragment thereof described herein for the effective treatment of the colorectal cancer. Other assays, e.g., urine tests, blood tests, ultrasound, X-rays, or cystoscopy, can also be used to monitor treatment in a patient, or to detect the presence, e.g., decreased presence (or absence), of a symptom of the disorder or disease, e.g., the CTHRC1-associated disorder or disease and/or CTHRC1-associated cancer, after treatment of the disorder or disease in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder or disease, e.g., a CTHRC1-associated disorder or disease and/or CTHRC1-associated cancer. In an embodiment, treatment is of a subject who does not exhibit certain signs of a disorder or disease, e.g., a CTHRC1-associated disorder or disease and/or CTHRC1-associated cancer, and/or of a subject who exhibits only early signs of a disorder or disease, e.g., a CTHRC1-associated disorder or disease and/or CTHRC1-associated cancer. In an embodiment, treatment is of a subject who exhibits one or more established signs of a disorder or disease, e.g., a CTHRC1-associated disorder or disease and/or CTHRC1-associated cancer. In an embodiment, treatment is of a subject diagnosed as suffering from a disorder or disease, e.g., a CTHRC1-associated disorder or disease and/or CTHRC1-associated cancer. In an embodiment, the disorder or disease is a CTHRC1-associated disorder or disease and/or CTHRC1-associated cancer described herein.

As used herein, the term “prevent,” a disorder or disease, e.g., a CTHRC1-associated disorder or disease and/or CTHRC1-associated cancer, means that a subject (e.g., a human) is less likely to have or develop the disorder or disease, e.g., a CTHRC1-associated disorder or disease and/or CTHRC1-associated cancer, if the subject receives the antibody, or antigen-binding fragment thereof. In an embodiment, the subject is at risk of developing the disorder or disease, e.g., a CTHRC1-associated disorder or disease and/or CTHRC1-associated cancer.

Various aspects of the compositions and methods herein are described in further detail below. Additional definitions are set out throughout the specification.

### Collagen Triple Helix Repeat Containing 1 (CTHRC1)

CTHRC1 (collagen triple helix repeat containing 1), is a protein that in humans is encoded by the CTHRC1 gene. CTHRC1 is a secreted, water-soluble protein involved in vascular remodeling, bone formation, and morphogenesis, and it is known that the protein is expressed in tissues undergoing repair such as injured arteries, skin wounds, and infarcted hearts where it contributes to tissue remodeling by modulating collagen synthesis in fibroblasts. Additionally, CTHRC1 increases bone formation by inhibiting osteoclast differentiation. Based on these, the role of CTHRC1 in the regulation of tissue repair and remodeling can be confirmed.

Exemplary amino acid and nucleotide sequences of human CTHRC1 are described, e.g., in SEQ ID NO: 37.

The amino acid sequence of human CTHRC1 is provided as follows.

Human CTHRC1
(SEQ ID NO: 37)
MRPQGPAASPQRLRGLLLLLLLQLPAPSSASEIPKGKQKAQLRQREVVDL
YNGMCLQGPAGVPGRDGSPGANGIPGTPGIPGRDGFKGEKGECLRESFEE
SWTPNYKQCSWSSLNYGIDLGKIAECTFTKMRSNSALRVLFSGSLRLKCR
NACCQRWYFTFNGAECSGPLPIEAIIYLDQGSPEMNSTINIHRTSSVEGL
CEGIGAGLVDVAIWVGTCSDYPKGDASTGWNSVSRIIIEELPK

Other variant and alternative sequences of human CTHRC1 are provided, e.g., as Genbank Accession Nos. NP_001243028.1 (isoform 2), NP_612464.1 (isoform 1 precursor), as listed as of Jan. 4, 2025. The respective nucleotide sequences encoding the above-listed human CTHRC1 sequences are provided as Genbank Accession Nos. NM 001256099.2 and NM_138455.4, as listed as of Jan. 4, 2025.

Exemplary amino acid and nucleotide sequences of mouse Cthrc1 are described, e.g., in Genbank Accession Nos. NP_081054.1 and NC_000081.7, respectively, as listed as of Jan. 14, 2025.

The amino acid sequence of mouse CTHRC1 is provided as follows.

Mouse CTHRC1
(SEQ ID NO: 38)
MHPQGRAAPPQLLLGLFLVLLLLLQLSAPISASENPKVKQKALIRQREVV
DLECTFTKMRSNSALRVLFSGSLRLKCRNACCQRWYFTFNGAECSGPLPI
EAIIYLDQGSPELNSTINIHRTSSVEGLCEGIGAGLVDVAIWVGTCSDYP
KGDASTGWNSVSRIIIEELPK

In an embodiment, when an anti-CTHRC1 antibody, or antigen-binding fragment thereof binds, or substantially binds, to CTHRC1, it binds, or substantially binds, to one or more isoforms of CTHRC1, e.g., one or more isoforms of human CTHRC1 described herein. In an embodiment, the antibody, or antigen-binding fragment thereof binds or substantially binds to CTHRC1 having the amino acid sequence of SEQ ID NO: 37. In an embodiment, the antibody, or antigen-binding fragment thereof binds or substantially binds to CTHRC1 having an amino acid sequence comprising at least 80%, 85%, 90%, 91%, 92%, 93%, 94%, 95%, 96%, 97%, 98%, 99%, or 99.9% of the amino acid sequence set forth in SEQ ID NO: 37.

In an embodiment, when an anti-CTHRC1 antibody, or antigen-binding fragment thereof binds, or substantially binds, to CTHRC1, it binds, or substantially binds, to one or more isoforms of CTHRC1, e.g., one or more isoforms of mouse CTHRC1 described herein. In an embodiment, the antibody, or antigen-binding fragment thereof binds or substantially binds to CTHRC1 having the amino acid sequence of SEQ ID NO: 38. In an embodiment, the antibody, or antigen-binding fragment thereof binds or substantially binds to CTHRC1 having an amino acid sequence comprising at least 80%, 85%, 90%, 91%, 92%, 93%, 94%, 95%, 96%, 97%, 98%, 99%, or 99.9% of the amino acid sequence set forth in SEQ ID NO: 38.

### Antibody, or Antigen-Binding Fragment Thereof

Disclosed herein are antibody, or antigen-binding fragment thereof that bind to CTHRC1, e.g., an anti-CTHRC1 antibody, or antigen-binding fragment thereof described herein.

As used herein, the term “antibody, or antigen-binding fragment thereof” refers to a protein, e.g., an immunoglobulin chain or a fragment thereof, comprising at least one immunoglobulin variable domain sequence. The term “antibody, or antigen-binding fragment thereof” includes, for example, a full-length antibody and an antigen-binding fragment of an antibody.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, K and 2; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

For example, an antibody, or antigen-binding fragment thereof can include a heavy (H) chain variable domain sequence (abbreviated herein as VH), and a light (L) chain variable domain sequence (abbreviated herein as VL). In another example, an antibody, or antigen-binding fragment thereof includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv or sc(Fv)2, for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody, or antigen-binding fragment thereof can be monoclonal or polyclonal. In embodiments, the antibody, or antigen-binding fragment thereof is a whole IgG antibody. The antibody, or antigen-binding fragment thereof can also be a human, humanized, CDR-grafted, or in vitro generated antibody. The antibody, or antigen-binding fragment thereof can have a heavy chain constant region chosen from, e.g., IgG1, IgG2, IgG3, IgG4, or a chimera of two or more isotypes. The antibody, or antigen-binding fragment thereof can also have a light chain chosen from, e.g., kappa or lambda. The term “immunoglobulin” (Ig) is used interchangeably with the term “antibody” herein. In embodiments, the antibody, or antigen-binding fragment thereof is a multispecific antibody, or antigen-binding fragment thereof (e.g., a bispecific antibody, or antigen-binding fragment thereof).

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

In an embodiment, the antibody, or antigen-binding fragment thereof is a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In an embodiment, the antibody, or antigen-binding fragment thereof is a single domain antibody. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In an embodiment, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also within the scope of the invention.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3). As used herein, the terms “framework,” “FW” and “FR” are used interchangeably.

The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The definition of CDRs by IMGT®, the International ImMunoGeneTics Information System® (www.imgt.org), defines the IMGT number for all immunoglobulins and T cell receptor V regions of all species. (IMGT®, International ImmunoGeneTics Information System®; Lefranc M P et al., (1999) Nucleic Acids Res, 27 (1): 209-12; Ruiz M et al., (2000) Nucleic Acids Res, 28 (1): 219-21; Lefranc M P (2001) Nucleic Acids Res, 29 (1): 207-9; Lefranc M P (2003) Nucleic Acids Res, 31 (1): 307-10 Lefranc M P et al., (2005) Dev Comp Immunol, 29 (3): 185-203; Kaas Q et al., (2007) Briefings in Functional Genomics & Proteomics, 6 (4): 253-64). The methods used herein may utilize CDRs defined according to any of these systems, although preferred embodiments use Kabat, Chothia or IMGT defined CDRs. Under all definitions, each VH and VL typically includes three CDRs and four FRs, arranged from amino-terminus to carboxy-terminus in the following order: FR1, CDR1, FR2, CDR2, FR3, CDR3, FR4.

For example, under Kabat, the CDR amino acid residues in the heavy chain variable domain (VH) for 13E09 are numbered SEQ ID NO: 5 (HCDR1), SEQ ID NO: 6 (HCDR2), and SEQ ID NO: 7 (HCDR3); and the CDR amino acid residues in the light chain variable domain (VL) are numbered SEQ ID NO: 8 (LCDR1), SEQ ID NO: 9 (LCDR2), and SEQ ID NO: 10 (LCDR3). Under IMGT, the amino acids in the VH are numbered SEQ ID NO: 11 (HCDR1), SEQ ID NO: 12 (HCDR2), and SEQ ID NO: 13 (HCDR3); and the amino acid residues in VL are numbered SEQ ID NO: 14 (LCDR1), amino acid sequence “LAS” (LCDR2), and SEQ ID NO: 10 (LCDR3). Under Chothia, the CDR amino acids in the VH are numbered SEQ ID NO: 15 (HCDR1), SEQ ID NO: 16 (HCDR2), and SEQ ID NO: 7 (HCDR3); and the amino acid residues in VL are numbered SEQ ID NO: 8 (LCDR1), SEQ ID NO: 9 (LCDR2), and SEQ ID NO: 10 (LCDR3). By combining the CDR definitions of Kabat, IMGT and Chothia, the CDRs consist of amino acid residues SEQ ID NO: 5, SEQ ID NO: 11, SEQ ID NO: 15 (HCDR1), respectively, SEQ ID NO: 6, SEQ ID NO: 12, SEQ ID NO: 16 (HCDR2) respectively, and SEQ ID NO: 7 (Kabat and Chothia), SEQ ID NO: 13 (IMGT) (HCDR3) in human VH and amino acid residues SEQ ID NO: 8 (Kabat and Chothia), SEQ ID NO: 14 (IMGT) (LCDR1), SEQ ID NO: 9 (Kabat and Chothia), amino acid sequence “LAS” (IMGT) (LCDR2), and SEQ ID NO: 10 (Kabat, IMGT, and Chothia) (LCDR3) in human VL.

In an embodiment, the antibody, or antigen-binding fragment thereof comprises a VH, wherein the VH comprises three heavy chain complementarity determining regions (HCDR1, HCDR2, and HCDR3), wherein the VH comprises one, two, or all of the following: (a) an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 13E09 (e.g., amino acid sequence “TYWMH”, SEQ ID NO: 5); (b) an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 13E09 (e.g., amino acid sequence “MNDPANSEIRLNQKFKD”, SEQ ID NO: 6); or (c) an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 13E09 (e.g., amino acid sequence “WIYGSGYALDY”, SEQ ID NO: 7), wherein the HCDR1, HCDR2, and HCDR3 are according to Kabat numbering.

In an embodiment, the antibody, or antigen-binding fragment thereof comprises a VL, wherein the VL comprises three light chain complementarity determining regions (LCDR1, LCDR2, and LCDR3), wherein the light chain variable region comprises one, two, or all of the following: (a) an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 13E09 (e.g., amino acid sequence “RASKSVSVSGYSYMH”, SEQ ID NO: 8); (b) an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 13E09 (e.g., amino acid sequence “LASNLES”, SEQ ID NO: 9); or (c) an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 13E09 (e.g., amino acid sequence “QLSRELPYT”, SEQ ID NO: 10), wherein the LCDR1, LCDR2, and LCDR3 are according to Kabat numbering.

In an embodiment, the antibody, or antigen-binding fragment thereof comprises: (a) a VH comprising one, two, or all of the following: an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 13E09 (e.g., amino acid sequence “TYWMH”, SEQ ID NO: 5); an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 13E09 (e.g., amino acid sequence “MNDPANSEIRLNQKFKD”, SEQ ID NO: 6); or an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 13E09 (e.g., amino acid sequence “WIYGSGYALDY”, SEQ ID NO: 7); and (b) a VL comprising one, two, or all of the following: an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 13E09 (e.g., amino acid sequence “RASKSVSVSGYSYMH”, SEQ ID NO: 8); an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 13E09 (e.g., amino acid sequence “LASNLES”, SEQ ID NO: 9); or an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to Kabat numbering.

In an embodiment, the antibody, or antigen-binding fragment thereof comprises: (a) a VH comprising: an HCDR1 comprising the amino acid sequence of the HCDR1 of antibody 13E09 (e.g., amino acid sequence “TYWMH”, SEQ ID NO: 5); an HCDR2 comprising the amino acid sequence of the HCDR2 of antibody 13E09 (e.g., amino acid sequence “MNDPANSEIRLNQKFKD”, SEQ ID NO: 6); and an HCDR3 comprising the amino acid sequence of the HCDR3 of antibody 13E09 (e.g., amino acid sequence “WIYGSGYALDY”, SEQ ID NO: 7); and (b) a VL comprising: an LCDR1 comprising the amino acid sequence of the LCDR1 of antibody 13E09 (e.g., amino acid sequence “RASKSVSVSGYSYMH”, SEQ ID NO: 8); an LCDR2 comprising the amino acid sequence of the LCDR2 of antibody 13E09 (e.g., amino acid sequence “LASNLES”, SEQ ID NO: 9); and an LCDR3 comprising the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to Kabat numbering.

In an embodiment, the antibody, or antigen-binding fragment thereof comprises a VH, wherein the VH comprises three heavy chain complementarity determining regions (HCDR1, HCDR2, and HCDR3), wherein the VH comprises one, two, or all of the following: (a) an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 7H11 (e.g., amino acid sequence “NYWIH”, SEQ ID NO: 20); (b) an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 7H11 (e.g., amino acid sequence “SIYPGSGIIKYDEKFKS”, SEQ ID NO: 21); or (c) an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 7H11 (e.g., amino acid sequence “NADGYYGDYAMDY”, SEQ ID NO: 22), wherein the HCDR1, HCDR2, and HCDR3 are according to Kabat numbering.

In an embodiment, the antibody, or antigen-binding fragment thereof comprises a VL, wherein the VL comprises three light chain complementarity determining regions (LCDR1, LCDR2, and LCDR3), wherein the light chain variable region comprises one, two, or all of the following: (a) an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 7H11 (e.g., amino acid sequence “SASSSVSYMH”, SEQ ID NO: 23); (b) an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 7H11 (e.g., amino acid sequence “TTSNLAS”, SEQ ID NO: 24); or (c) an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 7H11 (e.g., amino acid sequence “QQRNSYPFT”, SEQ ID NO: 25), wherein the LCDR1, LCDR2, and LCDR3 are according to Kabat numbering.

In an embodiment, the antibody, or antigen-binding fragment thereof comprises: (a) a VH comprising one, two, or all of the following: an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 7H11 (e.g., amino acid sequence “NYWIH”, SEQ ID NO: 20); an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 7H11 (e.g., amino acid sequence “SIYPGSGIIKYDEKFKS”, SEQ ID NO: 21); or an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 7H11 (e.g., amino acid sequence “NADGYYGDYAMDY”, SEQ ID NO: 22); and (b) a VL comprising one, two, or all of the following: an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 7H11 (e.g., amino acid sequence “SASSSVSYMH”, SEQ ID NO: 23); an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 7H11 (e.g., amino acid sequence “TTSNLAS”, SEQ ID NO: 24); or an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to Kabat numbering.

In an embodiment, the antibody, or antigen-binding fragment thereof comprises: (a) a VH comprising: an HCDR1 comprising the amino acid sequence of the HCDR1 of antibody 7H11 (e.g., amino acid sequence “NYWIH”, SEQ ID NO: 20); an HCDR2 comprising the amino acid sequence of the HCDR2 of antibody 7H11 (e.g., amino acid sequence “SIYPGSGIIKYDEKFKS”, SEQ ID NO: 21); and an HCDR3 comprising the amino acid sequence of the HCDR3 of antibody 7H11 (e.g., amino acid sequence “NADGYYGDYAMDY”, SEQ ID NO: 22); and (b) a VL comprising: an LCDR1 comprising the amino acid sequence of the LCDR1 of antibody 7H11 (e.g., amino acid sequence “SASSSVSYMH”, SEQ ID NO: 23); an LCDR2 comprising the amino acid sequence of the LCDR2 of antibody 7H11 (e.g., amino acid sequence “TTSNLAS”, SEQ ID NO: 24); and an LCDR3 comprising the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to Kabat numbering.

In an embodiment, the antibody, or antigen-binding fragment thereof comprises a VH, wherein the VH comprises three heavy chain complementarity determining regions (HCDR1, HCDR2, and HCDR3), wherein the VH comprises one, two, or all of the following: (a) an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 13E09 (e.g., amino acid sequence “GYTFTTYW”, SEQ ID NO: 11); (b) an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 13E09 (e.g., amino acid sequence “NDPANSEI”, SEQ ID NO: 12); or (c) an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 13E09 (e.g., amino acid sequence “ARWIYGSGYALDY”, SEQ ID NO: 13), wherein the HCDR1, HCDR2, and HCDR3 are according to IMGT numbering.

In an embodiment, the antibody, or antigen-binding fragment thereof comprises a VL, wherein the VL comprises three light chain complementarity determining regions (LCDR1, LCDR2, and LCDR3), wherein the light chain variable region comprises one, two, or all of the following: (a) an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 13E09 (e.g., amino acid sequence “KSVSVSGYSY”, SEQ ID NO: 14); (b) an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 13E09 (e.g., amino acid sequence “LAS”); or (c) an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 13E09 (e.g., amino acid sequence “QLSRELPYT”, SEQ ID NO: 10), wherein the LCDR1, LCDR2, and LCDR3 are according to IMGT numbering.

In an embodiment, the antibody, or antigen-binding fragment thereof comprises: (a) a VH comprising one, two, or all of the following: an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 13E09 (e.g., amino acid sequence “GYTFTTYW”, SEQ ID NO: 11); an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 13E09 (e.g., amino acid sequence “NDPANSEI”, SEQ ID NO: 12); or an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 13E09 (e.g., amino acid sequence “ARWIYGSGYALDY”, SEQ ID NO: 13); and (b) a VL comprising one, two, or all of the following: an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 13E09 (e.g., amino acid sequence “KSVSVSGYSY”, SEQ ID NO: 14); an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 13E09 (e.g., amino acid sequence “LAS”); or an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to IMGT numbering.

In an embodiment, the antibody, or antigen-binding fragment thereof comprises: (a) a VH comprising: an HCDR1 comprising the amino acid sequence of the HCDR1 of antibody 13E09 (e.g., amino acid sequence “GYTFTTYW”, SEQ ID NO: 11); an HCDR2 comprising the amino acid sequence of the HCDR2 of antibody 13E09 (e.g., amino acid sequence “NDPANSEI”, SEQ ID NO: 12); and an HCDR3 comprising the amino acid sequence of the HCDR3 of antibody 13E09 (e.g., amino acid sequence “ARWIYGSGYALDY”, SEQ ID NO: 13); and (b) a VL comprising: an LCDR1 comprising the amino acid sequence of the LCDR1 of antibody 13E09 (e.g., amino acid sequence “KSVSVSGYSY”, SEQ ID NO: 14); an LCDR2 comprising the amino acid sequence of the LCDR2 of antibody 13E09 (e.g., amino acid sequence “LAS”); and an LCDR3 comprising the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to IMGT numbering.

In an embodiment, the antibody, or antigen-binding fragment thereof comprises a VH, wherein the VH comprises three heavy chain complementarity determining regions (HCDR1, HCDR2, and HCDR3), wherein the VH comprises one, two, or all of the following: (a) an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 7H11 (e.g., amino acid sequence “GYTFTNYW”, SEQ ID NO: 44); (b) an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 7H11 (e.g., amino acid sequence “IYPGSGII”, SEQ ID NO: 45); or (c) an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 7H11 (e.g., amino acid sequence “TRNADGYYGDYAMDY”, SEQ ID NO: 39), wherein the HCDR1, HCDR2, and HCDR3 are according to IMGT numbering.

In an embodiment, the antibody, or antigen-binding fragment thereof comprises a VL, wherein the VL comprises three light chain complementarity determining regions (LCDR1, LCDR2, and LCDR3), wherein the light chain variable region comprises one, two, or all of the following: (a) an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 7H11 (e.g., amino acid sequence “SSVSY”, SEQ ID NO: 40); (b) an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 7H11 (e.g., amino acid sequence “TT”); or (c) an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 7H11 (e.g., amino acid sequence “QQRNSYPFT”, SEQ ID NO: 25), wherein the LCDR1, LCDR2, and LCDR3 are according to IMGT numbering.

In an embodiment, the antibody, or antigen-binding fragment thereof comprises: (a) a VH comprising one, two, or all of the following: an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 7H11 (e.g., amino acid sequence “GYTFTNYW”, SEQ ID NO: 44); an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 7H11 (e.g., amino acid sequence “IYPGSGII”, SEQ ID NO: 45); or an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 7H11 (e.g., amino acid sequence “TRNADGYYGDYAMDY”, SEQ ID NO: 39); and (b) a VL comprising one, two, or all of the following: an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 7H11 (e.g., amino acid sequence “SSVSY”, SEQ ID NO: 40); an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 7H11 (e.g., amino acid sequence “TT”); or an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to IMGT numbering.

In an embodiment, the antibody, or antigen-binding fragment thereof comprises: (a) a VH comprising: an HCDR1 comprising the amino acid sequence of the HCDR1 of antibody 7H11 (e.g., amino acid sequence “GYTFTNYW”, SEQ ID NO: 44); an HCDR2 comprising the amino acid sequence of the HCDR2 of antibody 7H11 (e.g., amino acid sequence “TYPGSGII”, SEQ ID NO: 45); and an HCDR3 comprising the amino acid sequence of the HCDR3 of antibody 7H11 (e.g., amino acid sequence “TRNADGYYGDYAMDY”, SEQ ID NO: 39); and (b) a VL comprising: an LCDR1 comprising the amino acid sequence of the LCDR1 of antibody 7H11 (e.g., amino acid sequence “SSVSY”, SEQ ID NO: 40); an LCDR2 comprising the amino acid sequence of the LCDR2 of antibody 7H11 (e.g., amino acid sequence “TT”); and an LCDR3 comprising the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to IMGT numbering.

In an embodiment, the antibody, or antigen-binding fragment thereof comprises a VH, wherein the VH comprises three heavy chain complementarity determining regions (HCDR1, HCDR2, and HCDR3), wherein the VH comprises one, two, or all of the following: (a) an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 13E09 (e.g., amino acid sequence “GYTFTTY”, SEQ ID NO: 15); (b) an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 13E09 (e.g., amino acid sequence “DPANSE”, SEQ ID NO: 16); or (c) an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 13E09 (e.g., amino acid sequence “WIYGSGYALDY”, SEQ ID NO: 7), wherein the HCDR1, HCDR2, and HCDR3 are according to Chothia numbering.

In an embodiment, the antibody, or antigen-binding fragment thereof comprises a VL, wherein the VL comprises three light chain complementarity determining regions (LCDR1, LCDR2, and LCDR3), wherein the light chain variable region comprises one, two, or all of the following: (a) an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 13E09 (e.g., amino acid sequence “RASKSVSVSGYSYMH”, SEQ ID NO: 8); (b) an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 13E09 (e.g., amino acid sequence “LASNLES”, SEQ ID NO: 9); or (c) an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 13E09 (e.g., amino acid sequence “QLSRELPYT”, SEQ ID NO: 10), wherein the LCDR1, LCDR2, and LCDR3 are according to Chothia numbering.

In an embodiment, the antibody, or antigen-binding fragment thereof comprises: (a) a VH comprising one, two, or all of the following: an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 13E09 (e.g., amino acid sequence “GYTFTTY”, SEQ ID NO: 15); an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 13E09 (e.g., amino acid sequence “DPANSE”, SEQ ID NO: 16); or an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 13E09 (e.g., amino acid sequence “WIYGSGYALDY”, SEQ ID NO: 7); and (b) a VL comprising one, two, or all of the following: an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 13E09 (e.g., amino acid sequence “RASKSVSVSGYSYMH”, SEQ ID NO: 8); an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 13E09 (e.g., amino acid sequence “LASNLES”, SEQ ID NO: 9); or an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to Chothia numbering.

In an embodiment, the antibody, or antigen-binding fragment thereof comprises: (a) a VH comprising: an HCDR1 comprising the amino acid sequence of the HCDR1 of antibody 13E09 (e.g., amino acid sequence “GYTFTTY”, SEQ ID NO: 15); an HCDR2 comprising the amino acid sequence of the HCDR2 of antibody 13E09 (e.g., amino acid sequence “DPANSE”, SEQ ID NO: 16); and an HCDR3 comprising the amino acid sequence of the HCDR3 of antibody 13E09 (e.g., amino acid sequence “WIYGSGYALDY”, SEQ ID NO: 7); and (b) a VL comprising: an LCDR1 comprising the amino acid sequence of the LCDR1 of antibody 13E09 (e.g., amino acid sequence “RASKSVSVSGYSYMH”, SEQ ID NO: 8); an LCDR2 comprising the amino acid sequence of the LCDR2 of antibody 13E09 (e.g., amino acid sequence “LASNLES”, SEQ ID NO: 9); and an LCDR3 comprising the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to Chothia numbering.

In an embodiment, the antibody, or antigen-binding fragment thereof comprises a VH, wherein the VH comprises three heavy chain complementarity determining regions (HCDR1, HCDR2, and HCDR3), wherein the VH comprises one, two, or all of the following: (a) an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 7H11 (e.g., amino acid sequence “GYTFTNY”, SEQ ID NO: 41); (b) an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 7H11 (e.g., amino acid sequence “YPGSGI”, SEQ ID NO: 42); or (c) an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 7H11 (e.g., amino acid sequence “NADGYYGDYAMDY”, SEQ ID NO: 22), wherein the HCDR1, HCDR2, and HCDR3 are according to Chothia numbering.

In an embodiment, the antibody, or antigen-binding fragment thereof comprises a VL, wherein the VL comprises three light chain complementarity determining regions (LCDR1, LCDR2, and LCDR3), wherein the light chain variable region comprises one, two, or all of the following: (a) an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 7H11 (e.g., amino acid sequence “SASSSVSYMH”, SEQ ID NO: 23); (b) an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 7H11 (e.g., amino acid sequence “TTSNLAS”, SEQ ID NO: 24); or (c) an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 7H11 (e.g., amino acid sequence “QQRNSYPFT”, SEQ ID NO: 25), wherein the LCDR1, LCDR2, and LCDR3 are according to Chothia numbering.

In an embodiment, the antibody, or antigen-binding fragment thereof comprises: (a) a VH comprising one, two, or all of the following: an HCDR1 comprising an amino acid sequence that differs by no more than 1, 2, or 3 amino acid residues from, or has at least 85%, 90%, 95%, 99% or 100% homology with, the amino acid sequence of the HCDR1 of antibody 7H11 (e.g., amino acid sequence “GYTFTNY”, SEQ ID NO: 41); an HCDR2 comprising an amino acid sequence that differs by no more than 1, 2, or 3 amino acid residues from, or has at least 85%, 90%, 95%, 99% or 100% homology with, the amino acid sequence of the HCDR2 of antibody 7H11 (e.g., amino acid sequence “YPGSGI”, SEQ ID NO: 42); or an HCDR3 comprising an amino acid sequence that differs by no more than 1, 2, or 3 amino acid residues from, or has at least 85%, 90%, 95%, 99% or 100% homology with, the amino acid sequence of the HCDR3 of antibody 7H11 (e.g., amino acid sequence “NADGYYGDYAMDY”, SEQ ID NO: 22); and (b) a VL comprising one, two, or all of the following: an LCDR1 comprising an amino acid sequence that differs by no more than 1, 2, or 3 amino acid residues from, or has at least 85%, 90%, 95%, 99% or 100% homology with, the amino acid sequence of the LCDR1 of antibody 7H11 (e.g., amino acid sequence “SASSSVSYMH”, SEQ ID NO: 23); an LCDR2 comprising an amino acid sequence that differs by no more than 1, 2, or 3 amino acid residues from, or has at least 85%, 90%, 95%, 99% or 100% homology with, the amino acid sequence of the LCDR2 of antibody 7H11 (e.g., amino acid sequence “TTSNLAS”, SEQ ID NO: 24); or an LCDR3 comprising an amino acid sequence that differs by no more than 1, 2, or 3 amino acid residues from, or has at least 85%, 90%, 95%, 99% or 100% homology with, the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to Chothia numbering.

In an embodiment, the antibody, or antigen-binding fragment thereof comprises: (a) a VH comprising: an HCDR1 comprising the amino acid sequence of the HCDR1 of antibody 7H11 (e.g., amino acid sequence “GYTFTNY”, SEQ ID NO: 41); an HCDR2 comprising the amino acid sequence of the HCDR2 of antibody 7H11 (e.g., amino acid sequence “YPGSGI”, SEQ ID NO: 42); and an HCDR3 comprising the amino acid sequence of the HCDR3 of antibody 7H11 (e.g., amino acid sequence “NADGYYGDYAMDY”, SEQ ID NO: 22); and (b) a VL comprising: an LCDR1 comprising the amino acid sequence of the LCDR1 of antibody 7H11 (e.g., amino acid sequence “SASSSVSYMH”, SEQ ID NO: 23); an LCDR2 comprising the amino acid sequence of the LCDR2 of antibody 7H11 (e.g., amino acid sequence “TTSNLAS”, SEQ ID NO: 24); and an LCDR3 comprising the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to Chothia numbering.

In an embodiment, the antibody, or antigen-binding fragment thereof comprises a VH comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VH of antibody 13E09 (e.g., SEQ ID NO: 1).

In an embodiment, the antibody, or antigen-binding fragment thereof comprises a VL comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VL of antibody 13E09 (e.g., SEQ ID NO: 3).

In an embodiment, the antibody, or antigen-binding fragment thereof comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VH of antibody 13E09 (e.g., SEQ ID NO: 1); and (ii) a VL comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VL of antibody 13E09 (e.g., SEQ ID NO: 3).

In an embodiment, the antibody, or antigen-binding fragment thereof comprises: (i) a VH comprising the amino acid sequence of the VH of antibody 13E09 (e.g., SEQ ID NO: 1); and (ii) a VL comprising the amino acid sequence of the VL of antibody 13E09 (e.g., SEQ ID NO: 3).

In an embodiment, the antibody, or antigen-binding fragment thereof comprises a VH comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VH of antibody 7H11 (e.g., SEQ ID NO: 17).

In an embodiment, the antibody, or antigen-binding fragment thereof comprises a VL comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VL of antibody 7H11 (e.g., SEQ ID NO: 19).

In an embodiment, the antibody, or antigen-binding fragment thereof comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VH of antibody 7H11 (e.g., SEQ ID NO: 17); and (ii) a VL comprising an amino acid sequence that differs by no more than 1, 2, 3, 4, 5, 6, 7, 8, 9, 10, 11, 12, 13, 14, or 15 amino acid residues from, or has at least 85%, 90%, 95%, 96%, 97%, 98%, 99%, or 100% homology with, the amino acid sequence of the VL of antibody 7H11 (e.g., SEQ ID NO: 19).

In an embodiment, the antibody, or antigen-binding fragment thereof comprises: (i) a VH comprising: an HCDR1 comprising the amino acid sequence of the HCDR1 of antibody 13E09 (e.g., amino acid sequence “TYWMH”, SEQ ID NO: 5); an HCDR2 comprising the amino acid sequence of the HCDR2 of antibody 13E09 (e.g., amino acid sequence “MNDPANSEIRLNQKFKD”, SEQ ID NO: 6); and an HCDR3 comprising the amino acid sequence of the HCDR3 of antibody 13E09 (e.g., amino acid sequence “WIYGSGYALDY”, SEQ ID NO: 7), and (ii) a VL comprising: an LCDR1 comprising the amino acid sequence of the LCDR1 of antibody 13E09 (e.g., amino acid sequence “RASKSVSVSGYSYMH”, SEQ ID NO: 8); an LCDR2 comprising the amino acid sequence of the LCDR2 of antibody 13E09 (e.g., amino acid sequence “LASNLES”, SEQ ID NO: 9); and an LCDR3 comprising the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to Kabat numbering.

In an embodiment, the antibody, or antigen-binding fragment thereof comprises: (i) a VH comprising: an HCDR1 comprising the amino acid sequence of the HCDR1 of antibody 7H11 (e.g., amino acid sequence “NYWIH”, SEQ ID NO: 20); an HCDR2 comprising the amino acid sequence of the HCDR2 of antibody 7H11 (e.g., amino acid sequence “SIYPGSGIIKYDEKFKS”, SEQ ID NO: 21); and an HCDR3 comprising the amino acid sequence of the HCDR3 of antibody 7H11 (e.g., amino acid sequence “NADGYYGDYAMDY”, SEQ ID NO: 22), and (ii) a VL comprising: an LCDR1 comprising the amino acid sequence of the LCDR1 of antibody 7H11 (e.g., amino acid sequence “SASSSVSYMH”, SEQ ID NO: 23); an LCDR2 comprising the amino acid sequence of the LCDR2 of antibody 7H11 (e.g., amino acid sequence “TTSNLAS”, SEQ ID NO: 24); and an LCDR3 comprising the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to Kabat numbering.

In an embodiment, the antibody, or antigen-binding fragment thereof comprises: (i) a VH comprising: an HCDR1 comprising the amino acid sequence of the HCDR1 of antibody 13E09 (e.g., amino acid sequence “GYTFTTYW”, SEQ ID NO: 11); an HCDR2 comprising the amino acid sequence of the HCDR2 of antibody 13E09 (e.g., amino acid sequence “NDPANSEI”, SEQ ID NO: 12); and an HCDR3 comprising the amino acid sequence of the HCDR3 of antibody 13E09 (amino acid sequence “ARWIYGSGYALDY”, e.g., SEQ ID NO: 13), and (ii) a VL comprising: an LCDR1 comprising the amino acid sequence of the LCDR1 of antibody 13E09 (e.g., amino acid sequence “KSVSVSGYSY”, SEQ ID NO: 14); an LCDR2 comprising the amino acid sequence of the LCDR2 of antibody 13E09 (e.g., amino acid sequence “LAS”); and an LCDR3 comprising the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to IMGT numbering.

In an embodiment, the antibody, or antigen-binding fragment thereof comprises: (i) a VH comprising: an HCDR1 comprising the amino acid sequence of the HCDR1 of antibody 7H11 (e.g., amino acid sequence “GYTFTNYW”, SEQ ID NO: 44); an HCDR2 comprising the amino acid sequence of the HCDR2 of antibody 7H11 (e.g., amino acid sequence “IYPGSGII”, SEQ ID NO: 45); and an HCDR3 comprising the amino acid sequence of the HCDR3 of antibody 7H11 (e.g., amino acid sequence “TRNADGYYGDYAMDY”, SEQ ID NO: 39), and (ii) a VL comprising: an LCDR1 comprising the amino acid sequence of the LCDR1 of antibody 7H11 (e.g., amino acid sequence “SSVSY”, SEQ ID NO: 40); an LCDR2 comprising the amino acid sequence of the LCDR2 of antibody 7H11 (e.g., amino acid sequence “TT”); and an LCDR3 comprising the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to IMGT numbering.

In an embodiment, the antibody, or antigen-binding fragment thereof comprises: (i) a VH comprising: an HCDR1 comprising the amino acid sequence of the HCDR1 of antibody 13E09 (e.g., amino acid sequence “GYTFTTY”, SEQ ID NO: 15); an HCDR2 comprising the amino acid sequence of the HCDR2 of antibody 13E09 (e.g., amino acid sequence “DPANSE”, SEQ ID NO: 16); and an HCDR3 comprising the amino acid sequence of the HCDR3 of antibody 13E09 (e.g., amino acid sequence “WIYGSGYALDY”, SEQ ID NO: 7), and (ii) a VL comprising: an LCDR1 comprising the amino acid sequence of the LCDR1 of antibody 13E09 (e.g., amino acid sequence “RASKSVSVSGYSYMH”, SEQ ID NO: 8); an LCDR2 comprising the amino acid sequence of the LCDR2 of antibody 13E09 (e.g., amino acid sequence “LASNLES”, SEQ ID NO: 9); and an LCDR3 comprising the amino acid sequence of the LCDR3 of antibody 13E09 (e.g., amino acid sequence “QLSRELPYT”, SEQ ID NO: 10), wherein the HCDR1, HCDR2, HCDR3, LCDR1, LCDR2, and LCDR3 are according to Chothia numbering.

In an embodiment, the antibody, or antigen-binding fragment thereof comprises: (i) a VH comprising: an HCDR1 comprising the amino acid sequence of the HCDR1 of antibody 7H11 (e.g., amino acid sequence “GYTFTNY”, SEQ ID NO: 41); an HCDR2 comprising the amino acid sequence of the HCDR2 of antibody 7H11 (e.g., amino acid sequence “YPGSGI”, SEQ ID NO: 42); and an HCDR3 comprising the amino acid sequence of the HCDR3 of antibody 7H11 (e.g., amino acid sequence “NADGYYGDYAMDY”, SEQ ID NO: 22), and (ii) a VL comprising: an LCDR1 comprising the amino acid sequence of the LCDR1 of antibody 7H11 (e.g., amino acid sequence “SASSSVSYMH”, SEQ ID NO: 23); an LCDR2 comprising the amino acid sequence of the LCDR2 of antibody 7H11 (e.g., amino acid sequence “TTSNLAS”, SEQ ID NO: 24); and an LCDR3 comprising the amino acid sequence of the LCDR3 of antibody 7H11 (e.g., amino acid sequence “QQRNSYPFT”, SEQ ID NO: 25), wherein the HCDR1, HCDR2, HCDR3, LCDR1, LCDR2, and LCDR3 are according to Chothia numbering.

In an embodiment, the antibody, or antigen-binding fragment thereof comprises: (i) a VH comprising the amino acid sequence of the VH of antibody 7H11 (e.g., SEQ ID NO: 17); and (ii) a VL comprising the amino acid sequence of the VL of antibody 7H11 (e.g., SEQ ID NO: 19).

In an embodiment, the antibody, or antigen-binding fragment thereof comprises a VH encoded by the nucleotide sequence of SEQ ID NO: 28 (or a nucleotide sequence substantially identical thereto) or a VL encoded by the nucleotide sequence of SEQ ID NO: 30 (or a nucleotide sequence substantially identical thereto), or both. In an embodiment, the antibody, or antigen-binding fragment thereof comprises a VH encoded by the nucleotide sequence of SEQ ID NO: 32 (or a nucleotide sequence substantially identical thereto) or a VL encoded by the nucleotide sequence of SEQ ID NO: 34 (or a nucleotide sequence substantially identical thereto), or both.

In an embodiment the antibody, or antigen-binding fragment thereof is antibody 13E09, or 7H11.

In an embodiment, the antibody, or antigen-binding fragment thereof comprises one or more framework regions derived from a human framework germline sequence.

In an embodiment, the antibody, or antigen-binding fragment thereof comprises two VHs and two VLs. In an embodiment, the antibody, or antigen-binding fragment thereof comprises an antigen-binding fragment. In an embodiment, the antibody, or antigen-binding fragment thereof comprises a Fab, F(ab′)2, Fv, scFv, or Fd.

In an embodiment, the antibody, or antigen-binding fragment thereof is an IgG antibody, or antigen-binding fragment thereof, e.g., comprising a heavy chain constant region of IgG, e.g., chosen from IgG1, IgG2, IgG3, or IgG4, e.g., IgG2 or IgG4. In an embodiment, the antibody, or antigen-binding fragment thereof is an IgG1 antibody, or antigen-binding fragment thereof, e.g., having an IgG1 constant region described herein. In another embodiment, the antibody, or antigen-binding fragment thereof is an IgG2 antibody, or antigen-binding fragment thereof e.g., having an IgG2 constant region described herein. In an embodiment, the antibody, or antigen-binding fragment thereof is an IgG3 antibody, or antigen-binding fragment thereof, e.g., having an IgG3 constant region described herein. In another embodiment, the antibody, or antigen-binding fragment thereof is an IgG4 antibody, or antigen-binding fragment thereof e.g., having an IgG4 constant region described herein. In another embodiment, the antibody, or antigen-binding fragment thereof has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody, or antigen-binding fragment thereof comprises a light chain constant region of kappa or lambda light chain.

In an aspect, the disclosure features an anti-CTHRC1 antibody, or antigen-binding fragment thereof described herein, e.g., a synthetic or isolated anti-CTHRC1 antibody, or antigen-binding fragment thereof described herein.

In an embodiment, the antibody, or antigen-binding fragment thereof is a synthetic antibody, or antigen-binding fragment thereof. In an embodiment, the antibody, or antigen-binding fragment thereof is an isolated antibody, or antigen-binding fragment thereof. In an embodiment, the antibody, or antigen-binding fragment thereof is a recombinant antibody, or antigen-binding fragment thereof. In an embodiment, the antibody, or antigen-binding fragment thereof is a humanized antibody. In an embodiment, the antibody, or antigen-binding fragment thereof is a mono-specific antibody, or antigen-binding fragment thereof. In an embodiment, the antibody, or antigen-binding fragment thereof is a multi-specific antibody, or antigen-binding fragment thereof.

In an embodiment, the antibody, or antigen-binding fragment thereof competes for binding with an anti-CTHRC1 antibody, or antigen-binding fragment thereof that comprises a VH and a VL of any of antibodies 13E09 or 7H11.

In an embodiment, the antibody, or antigen-binding fragment thereof binds, or substantially binds, to an epitope that completely or partially overlaps with the epitope of two, three, four, five, six, seven, or all of an anti-CTHRC1 antibody, or antigen-binding fragment thereof that comprises a VH and a VL of any of antibodies 13E09 or 7H11.

In an embodiment, the antibody, or antigen-binding fragment thereof competes for binding with the anti-CTHRC1 antibody, or antigen-binding fragment thereof that comprise the HCDR1, HCDR2, HCDR3, LCDR1, LCDR2, and LCDR3 of any of antibodies 13E09 and/or 7H11.

In an embodiment, the antibody, or antigen-binding fragment thereof competes for binding with the anti-CTHRC1 antibody, or antigen-binding fragment thereof that comprises a VH and a VL of any of antibodies 13E09 and/or 7H11.

In an embodiment, the antibody, or antigen-binding fragment thereof binds, or substantially binds, to an epitope that completely or partially overlaps with the epitopes of two, three, four, five, six, seven, or all of the anti-CTHRC1 antibody, or antigen-binding fragment thereof that comprise the HCDR1, HCDR2, HCDR3, LCDR1, LCDR2, and LCDR3 of any of antibodies 13E09 or 7H11.

Generally, unless specifically indicated, the anti-CTHRC1 antibody, or antigen-binding fragment thereof described herein can include any combination of one or more Kabat CDRs, IMGT CDRs, and/or Chothia hypervariable loops, e.g., described in Tables 1-2.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody, or antigen-binding fragment thereof that comprises determinants that form an interface that binds to an antigen, e.g., CTHRC1, e.g., human CTHRC1, or an epitope thereof. With respect to proteins (or protein mimetics), the antigen-binding region typically includes one or more loops (of at least, e.g., four amino acids or amino acid mimics) that form an interface that binds to the antigen, e.g., CTHRC1, e.g., human CTHRC1. Typically, the antigen-binding region of an antibody, or antigen-binding fragment thereof includes at least one or two CDRs and/or hypervariable loops, or more typically at least three, four, five or six CDRs and/or hypervariable loops.

The terms “specific binding” or “specifically binding”, as used herein, in reference to the interaction of an antibody, e.g., anti-CTHRC1 antibody, with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “compete” or “cross-compete” are used interchangeably herein to refer to the ability of an antibody, or antigen-binding fragment thereof to interfere with binding of an anti-CTHRC1 antibody, or antigen-binding fragment thereof, e.g., an anti-CTHRC1 antibody, or antigen-binding fragment thereof provided herein, to a target, e.g., CTHRC1, e.g., human CTHRC1. The interference with binding can be direct or indirect (e.g., through an allosteric modulation of the antibody, or antigen-binding fragment thereof or the target). The extent to which an antibody, or antigen-binding fragment thereof is able to interfere with the binding of another antibody, or antigen-binding fragment thereof to the target, and therefore whether it can be said to compete, can be determined using a competition binding assay, for example, a FACS assay, an ELISA, or a BIACORE assay. In an embodiment, a competition binding assay is a quantitative competition assay. In an embodiment, a first anti-CTHRC1 antibody, or antigen-binding fragment thereof is said to compete for binding to the target with a second anti-CTHRC1 antibody, or antigen-binding fragment thereof when the binding of the first antibody, or antigen-binding fragment thereof to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody, or antigen-binding fragment thereof of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody, or antigen-binding fragment thereof can be a polyclonal or a monoclonal antibody. In an embodiment, the antibody can be recombinantly produced, e.g., produced by any suitable phage display or combinatorial methods.

In an embodiment, the antibody, or antigen-binding fragment thereof is a fully human antibody (e.g., an antibody made in a mouse which has been genetically engineered to produce an antibody from a human immunoglobulin sequence), or a non-human antibody, e.g., a rodent (e.g., mouse or rat), goat, primate (e.g., monkey), camel antibody. In an embodiment, the non-human antibody is a rodent (e.g., mouse or rat antibody). Methods of producing rodent antibodies are known in the art.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Also provided are humanized antibodies in which specific amino acids have been substituted, deleted or added. Criteria for selecting amino acids from the donor are described in, e.g., U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In an embodiment, the antibody, or antigen-binding fragment thereof has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody, or antigen-binding fragment thereof has a light chain constant region chosen from, e.g., the (e.g., human) light chain constant regions of kappa or lambda. The constant region can be altered, e.g., mutated, to modify the properties of the antibody, or antigen-binding fragment thereof (e.g., to increase or decrease Fc receptor binding, antibody glycosylation, or the number of cysteine residues). In an embodiment, the antibody, or antigen-binding fragment thereof has reduced or no ability to bind an Fc receptor. For example, it may be an isotype or subtype, fragment or other mutant, which does not support binding to an Fc receptor, e.g., it has a mutated or deleted Fc receptor binding region.

In an embodiment, a constant region of the antibody, or antigen-binding fragment thereof is altered. Methods for altering an antibody constant region are known in the art. Antibody, or antigen-binding fragment thereof with altered function, e.g. altered affinity for an effector ligand, such as FcR on a cell can be produced by replacing at least one amino acid residue in the constant portion of the antibody with a different residue. Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

In an embodiment, the antibody, or antigen-binding fragment thereof comprises a monoclonal antibody (e.g., a full length antibody which has an immunoglobulin Fc region). In an embodiment, the antibody, or antigen-binding fragment thereof comprises a full length antibody or full length immunoglobulin chain. In an embodiment, the antibody, or antigen-binding fragment thereof comprises an antigen binding or functional fragment of a full length antibody or full length immunoglobulin chain.

In an embodiment, the antibody, or antigen-binding fragment thereof is a monospecific antibody, or antigen-binding fragment thereof, e.g., it binds a single epitope. For example, a monospecific antibody, or antigen-binding fragment thereof can have a plurality of immunoglobulin variable region sequences, each of which binds the same epitope.

In an embodiment, the antibody, or antigen-binding fragment thereof is a multispecific antibody, or antigen-binding fragment thereof, e.g., it comprises a plurality of immunoglobulin variable region sequences, wherein a first immunoglobulin variable region sequence of the plurality has binding specificity for a first epitope and a second immunoglobulin variable region sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody, or antigen-binding fragment thereof comprises a third, fourth or fifth immunoglobulin variable domain. In an embodiment, a multispecific antibody, or antigen-binding fragment thereof is a bispecific antibody, or antigen-binding fragment thereof, a trispecific antibody, or antigen-binding fragment thereof, or tetraspecific antibody, or antigen-binding fragment thereof.

In an embodiment, a multispecific antibody, or antigen-binding fragment thereof is a bispecific antibody, or antigen-binding fragment thereof. A bispecific antibody has specificity for no more than two antigens. A bispecific antibody, or antigen-binding fragment thereof is typically characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment, the first and second epitopes do not overlap. In an embodiment, the first and second epitopes are on different antigens, e.g., the different proteins (or different subunits of a multimeric protein). In an embodiment, a bispecific antibody, or antigen-binding fragment thereof comprises a heavy chain variable region sequence and a light chain variable region sequence which have binding specificity for a first epitope, and a heavy chain variable region sequence and a light chain variable region sequence which have binding specificity for a second epitope. In an embodiment, a bispecific antibody, or antigen-binding fragment thereof comprises a half antibody having binding specificity for a first epitope and a half antibody having binding specificity for a second epitope. In an embodiment, a bispecific antibody, or antigen-binding fragment thereof comprises a half antibody, or a fragment thereof, having binding specificity for a first epitope, and a half antibody, or fragment thereof, having binding specificity for a second epitope. In an embodiment a bispecific antibody, or antigen-binding fragment thereof comprises an scFv, or a fragment thereof, have binding specificity for a first epitope, and an scFv, or a fragment thereof, have binding specificity for a second epitope.

A polypeptide of an antibody, or antigen-binding fragment thereof described herein may be linear or branched, it may comprise modified amino acids, and it may be interrupted by non-amino acids. The antibody, or antigen-binding fragment thereof may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody, or antigen-binding fragment thereof described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-CTHRC1 antibody, or antigen-binding fragment thereof can be coupled to a radioactive isotope such as an α-, β-, or γ-emitter, or a β- and γ-emitter.

An antibody, or antigen-binding fragment thereof can be derivatized or linked to another functional molecule (e.g., another peptide or protein). As used herein, a “derivatized” antibody, or antigen-binding fragment thereof is one that has been modified. Methods of derivatization include but are not limited to the addition of a fluorescent moiety, a radionucleotide, a toxin, an enzyme or an affinity ligand such as biotin. Accordingly, the antibody, or antigen-binding fragment thereof is intended to include derivatized and otherwise modified forms of the antibodies described herein, including immunoadhesion molecules. For example, an antibody, or antigen-binding fragment thereof can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody, or antigen-binding fragment thereof are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-CTHRC1 antibody, or antigen-binding fragment thereof may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody, or antigen-binding fragment thereof may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody, or antigen-binding fragment thereof can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody, or antigen-binding fragment thereof described herein can be conjugated to another molecular entity, typically a label or a therapeutic (e.g., antimicrobial (e.g., antibacterial or bactericidal), immunomodulatory, immunostimularoty, cytotoxic, or cytostatic) agent or moiety.

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8. Non-limiting examples of drugs that may be included in the ADCs are mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The term “extracellular-targeted drug conjugate” or “EDC” refers to a drug conjugate of the invention in which an antibody or other targeting moiety that targets an extracellular target is linked via a stable or non-cleavable linker to a drug that binds to an extracellular target.

The term “extracellular” refers to proteins, antigens, or epitopes located on the external portion of a cell membrane or are in the extracellular matrix.

In an embodiment, the antibody, or antigen-binding fragment thereof is conjugated to a diagnostic agent, e.g., radioisotopes. Radioactive isotopes can be used in diagnostic or therapeutic applications. Radioactive isotopes that can be coupled to the antibody, or antigen-binding fragment thereof include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

In an embodiment, the antibody, or antigen-binding fragment thereof is conjugated to a therapeutic agent. Therapeutically active radioisotopes are disclosed herein. Examples of other therapeutic agents include, but are not limited to, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see e.g., U.S. Pat. No. 5,208,020), CC-1065 (see e.g., U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In an embodiment, the anti-CTHRC1 antibody, or antigen-binding fragment thereof (e.g., a monospecific, bispecific, or multispecific antibody, or antigen-binding fragment thereof) is covalently linked, e.g., fused, to another partner e.g., a protein, e.g., as a fusion molecule (e.g., a fusion protein).

As used herein, a “fusion protein” and “fusion polypeptide” refer to a polypeptide having at least two portions covalently linked together, where each of the portions is a polypeptide. In an embodiment, each of the portions is a polypeptide that has a different property. The property can be a biological property, such as activity in vitro or in vivo. The property can also be simple chemical or physical property, such as binding to a target molecule, catalysis of a reaction, etc. The two portions can be linked directly by a single peptide bond or through a linker (e.g., peptide linker), but are in reading frame with each other.

In one aspect, the invention features a method of providing a target binding agent that specifically binds to CTHRC1 (e.g., human CTHRC1). For example, the target binding molecule is an antibody, or antigen-binding fragment thereof. The method includes: providing a target protein that comprises at least a portion of non-human protein, the portion being homologous to (e.g., at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining a binding agent (e.g., an antibody, or antigen-binding fragment thereof) that specifically binds to the target protein; and evaluating efficacy of the binding agent in modulating an activity of the target protein. The method can further include administering the binding agent (e.g., antibody, or antigen-binding fragment thereof) or a derivative (e.g., a humanized antibody, or antigen-binding fragment thereof) to a subject (e.g., a human subject).

In another aspect, this disclosure provides a method of making an antibody, or antigen-binding fragment thereof disclosed herein. The method includes: providing an antigen, e.g., CTHRC1 (e.g., human CTHRC1) or a fragment thereof; obtaining an antibody, or antigen-binding fragment thereof that specifically binds to the antigen; evaluating efficacy of the antibody, or antigen-binding fragment thereof in modulating activity of the antigen and/or organism expressing the antigen, e.g., CTHRC1, e.g., human CTHRC1. The method can further include administering the antibody, or antigen-binding fragment thereof, including a derivative thereof (e.g., a humanized antibody, or antigen-binding fragment thereof) to a subject, e.g., a human.

This disclosure provides an isolated nucleic acid molecule encoding the above antibody, or antigen-binding fragment thereof, vectors and host cells thereof. The nucleic acid molecule includes, but is not limited to, RNA, genomic DNA and cDNA.

Amino acid and nucleotide sequences of exemplary antibody, or antigen-binding fragment thereof are described in Tables 1-5.

The term “monospecific antibody” refers to an antibody capable of binding only one antigen.

The term “bispecific antibody” refers to an antibody capable of binding two unrelated antigens.

The term “multispecific antibody” refers to an antibody capable of binding two or more unrelated antigens.

Another embodiment of the disclosure provides a glycosylated binding protein wherein the anti-CTHRC1 antibody or antigen-binding fragment thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993)30:1361-1367), or result in increased affinity for the antigen (Wallick, S. C., et al., Exp. Med. (1988)168:1099-1109; Wright, A., et al., EMBO J. (1991)10:2717-2723).

Antibodies may be produced by any of a number of techniques. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies disclosed herein include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Epitope

The antibody, or antigen-binding fragment thereof described herein can bind to an epitope on CTHRC1 (e.g., human CTHRC1, SEQ ID NO: 37). For example, an epitope bound by an antibody, or antigen-binding fragment thereof described herein can include one or more epitope contact points in a CTHRC1 protein sequence described herein.

### Animal Models

The antibody, or antigen-binding fragment thereof described herein can be evaluated in vivo, e.g., using various animal models. For example, an animal model can be used to test the efficacy of an antibody, or antigen-binding fragment thereof described herein treating or preventing a disorder or disease described herein, e.g., a CTHRC1-associated disorder or disease and/or CTHRC1-associated cancer. Animal models can also be used, e.g., to investigate for side effects, measure concentrations of antibody, or antigen-binding fragment thereof in situ, demonstrate correlations between a CTHRC1 function and a CTHRC1-associated disorder or disease and/or CTHRC1-associated cancer.

Exemplary animal models for a CTHRC1-associated disorder or disease and/or CTHRC1-associated cancer described herein that can be used for evaluating an antibody, or antigen-binding fragment thereof described herein include, but are not limited to, C57BL/6J subcutaneous colorectal tumor (MC38^{Luc+/RFP+})) syngeneic mouse model, and C57BL/6J Cthrc1 global KO mouse.

Exemplary animal models for other disorders or diseases described herein are also known in the art. Exemplary types of animals that can be used to evaluate the antibody, or antigen-binding fragment thereof described herein include, but are not limited to, mice, rats, rabbits, guinea pigs, and monkeys.

### Pharmaceutical Compositions and Kits

In an aspect, this disclosure provides compositions, e.g., pharmaceutically acceptable compositions, which include an antibody, or antigen-binding fragment thereof described herein (e.g., a humanized antibody, or antigen-binding fragment thereof described herein), formulated together with a pharmaceutically acceptable carrier. The pharmaceutical compositions comprising antibodies are for use in, but not limited to, diagnosing, detecting, or monitoring a disorder or disease, in preventing, treating, managing, or ameliorating of a disorder or disease, or one or more symptoms thereof, and/or in research. In a specific embodiment, a composition comprises one or more antibodies. In another embodiment, the pharmaceutical composition comprises one or more antibodies and one or more prophylactic or therapeutic agents other than antibodies for treating a disorder or disease in which CTHRC1 activity is detrimental. Preferably, the prophylactic or therapeutic agents known to be useful for or having been or currently being used in the prevention, treatment, management, or amelioration of a disorder or disease or one or more symptoms thereof. In accordance with these embodiments, the composition may further comprise of a carrier, diluent or excipient.

The antibodies and antibody-portions can be incorporated into pharmaceutical compositions suitable for administration to a subject. Typically, the pharmaceutical composition comprises an antibody or antibody portion and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion. The carrier can be suitable for intravenous, intramuscular, subcutaneous, parenteral, rectal, spinal or epidermal administration (e.g., by injection or infusion). In an embodiment, less than about 5%, e.g., less than about 4%, 3%, 2%, or 1% of the antibody, or antigen-binding fragment thereof in the pharmaceutical composition are present as aggregates. In other embodiments, at least about 95%, e.g., at least about 96%, 97%, 98%, 98.5%, 99%, 99.5%, 99.8%, or more of the antibody, or antigen-binding fragment thereof in the pharmaceutical composition are present as monomers. In an embodiment, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In an embodiment, the antibody, or antigen-binding fragment thereof is administered by intravenous infusion or injection. In an embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody, or antigen-binding fragment thereof described herein can be administered by a variety of methods. Several are known in the art, and for many therapeutic, prophylactic, or diagnostic applications, an appropriate route/mode of administration is intravenous injection or infusion. For example, the antibody, or antigen-binding fragment thereof can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In an embodiment, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In an embodiment, an antibody, or antigen-binding fragment thereof can be orally administered, for example, with an inert diluent or an assimilable edible carrier. The antibody, or antigen-binding fragment thereof (and other ingredients, if desired) may also be enclosed in a hard or soft shell gelatin capsule, compressed into tablets, or incorporated directly into the subject's diet. For oral therapeutic administration, the antibody, or antigen-binding fragment thereof may be incorporated with excipients and used in the form of ingestible tablets, buccal tablets, troches, capsules, elixirs, suspensions, syrups, wafers, and the like. To administer an antibody, or antigen-binding fragment thereof by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody, or antigen-binding fragment thereof and the particular therapeutic, prophylactic, or diagnostic effect to be achieved, and (b) the limitations inherent in the art of compounding such an antibody, or antigen-binding fragment thereof for the treatment of sensitivity in individuals.

An exemplary, non-limiting range for a therapeutically, prophylactically, or diagnostically effective amount of an antibody, or antigen-binding fragment thereof is about 0.1-50 mg/kg body weight of a subject, e.g., about 0.1-30 mg/kg, e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount,” or “diagnostically effectively amount” of an antibody, or antigen-binding fragment thereof described herein.

A “therapeutically effective amount” refers to an amount effective, at dosages and for periods of time necessary, to achieve the desired therapeutic result. A therapeutically effective amount of the antibody, or antigen-binding fragment thereof may vary according to factors such as the disease state, age, sex, and weight of the individual, and the ability of the antibody or antibody portion to elicit a desired response in the individual. A therapeutically effective amount is also one in which any toxic or detrimental effect of the antibody, or antigen-binding fragment thereof is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody, or antigen-binding fragment thereof to inhibit a measurable parameter can be evaluated in an animal model system predictive of efficacy in treating or preventing IgA nephropathy. Alternatively, this property of a composition can be evaluated by examining the ability of the antibody, or antigen-binding fragment thereof to reduce tumor burden, e.g., by an in vivo study, e.g., by measuring whole mouse tumor burden.

A “prophylactically effective amount” refers to an amount effective, at dosages and for periods of time necessary, to achieve the desired prophylactic result. Typically, since a prophylactic dose is used in subjects prior to or at an earlier stage of disease or disorder, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder or disease, e.g., a disorder or disease described herein, e.g., IgA nephropathy, can be diagnosed in vitro, ex vivo, or in vivo.

Also within this disclosure is a kit that comprises an antibody, or antigen-binding fragment thereof, described herein. The kit can include one or more other elements including: instructions for use; other reagents, e.g., a label, a therapeutic agent, or an agent useful for chelating, or otherwise coupling, an antibody, or antigen-binding fragment thereof to a label or therapeutic agent, or a radioprotective composition; devices or other materials for preparing the antibody, or antigen-binding fragment thereof for administration; pharmaceutically acceptable carriers; and devices or other materials for administration to a subject.

### Nucleic Acids

The present disclosure also features nucleic acids comprising nucleotide sequences that encode the antibody, or antigen-binding fragment thereof (e.g., heavy and light chain variable regions and CDRs of the antibody, or antigen-binding fragment thereof), as described herein.

For example, the present disclosure features a first and second nucleic acid encoding heavy and light chain variable regions, respectively, of an antibody, or antigen-binding fragment thereof chosen from one or more of the antibodies, or antigen-binding fragments thereof disclosed herein, e.g., an antibody, or antigen-binding fragment thereof of Tables 1-2 or a portion of an antibody, or antigen-binding fragment thereof, e.g., the variable regions of Tables 1-2. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein).

In an embodiment, the nucleic acid can comprise a nucleotide sequence encoding at least one, two, or three CDRs from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, or three CDRs from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, three, four, five, or six CDRs from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In an embodiment, the nucleic acid can comprise a nucleotide sequence encoding at least one, two, or three CDRs from a heavy chain variable region having the nucleotide sequence as set forth in Table 4, a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, or three CDRs from a light chain variable region having the nucleotide sequence as set forth in Table 4, or a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, three, four, five, or six CDRs from heavy and light chain variable regions having the nucleotide sequence as set forth in Table 4, or a sequence substantially homologous thereto (e.g., a sequence at least about 85%, 90%, 95%, 99% or more identical thereto, and/or capable of hybridizing under the stringency conditions described herein).

The portion may encode, for example, a variable region (e.g., VH or VL); one, two, or three or more CDRs; or one, two, three, or four or more framework regions.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In an aspect, the application features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an antibody, or antigen-binding fragment thereof described herein.

In an embodiment, the vector comprises a nucleotide encoding an antibody, or antigen-binding fragment thereof described herein, e.g., as described in Tables 1-2. In another embodiment, the vector comprises a nucleotide sequence described herein, e.g., in Table 4. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody, or antigen-binding fragment thereof produced are known to those skilled in the art, and may be varied or optimized depending upon the specific expression vector and mammalian host cell employed, based upon the present description.

### Uses of Antibody, or Antigen-Binding Fragment Thereof

The antibody, or antigen-binding fragment thereof disclosed herein, as well as the pharmaceutical compositions disclosed herein, have in vitro, ex vivo, and in vivo therapeutic, prophylactic, and/or diagnostic utilities.

In an embodiment, the antibody, or antigen-binding fragment thereof reduces (e.g., inhibits, blocks, or neutralizes) one or more biological activities of CTHRC1. For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of CTHRC1. Accordingly, in an aspect, the disclosure provides a method of treating, preventing, or diagnosing a disorder or disease, e.g., a disorder or disease described herein (e.g., colorectal cancer), in a subject, comprising administering to the subject an antibody, or antigen-binding fragment thereof described herein, such that the disorder or disease is treated, prevented, or diagnosed. For example, the disclosure provides a method comprising contacting the antibody, or antigen-binding fragment thereof described herein with cells in culture, e.g. in vitro or ex vivo, or administering the antibody, or antigen-binding fragment thereof described herein to a subject, e.g., in vivo, to treat, prevent, or diagnose a disease, e.g., a disorder or disease associated with CTHRC1, e.g., a CTHRC1-associated disorder or disease and/or CTHRC1-associated cancer described herein.

The disclosure provides methods for reducing CTHRC1 activity in a subject suffering from such a disease or disorder, which method comprises administering to the subject an antibody or antibody portion of the disclosure such that CTHRC1 activity in the subject is reduced. Preferably, the CTHRC1 is human CTHRC1, and the subject is a human subject. Alternatively, the subject can be a mammal expressing a CTHRC1 to which antibodies of the disclosure are capable of binding. Still further the subject can be a mammal into which CTHRC1 has been introduced (e.g., by administration of CTHRC1 or by expression of a CTHRC1 transgene). Antibodies of the disclosure can be administered to a human subject for therapeutic purposes. Moreover, antibodies of the disclosure can be administered to a non-human mammal expressing a CTHRC1 with which the antibody is capable of binding for veterinary purposes or as an animal model of human disease or disorder. Regarding the latter, such animal models may be useful for evaluating the therapeutic efficacy of antibodies of the disclosure (e.g., testing of dosages and time courses of administration).

As used herein, the term “subject” is intended to include human and non-human animals. In an embodiment, the subject is a human subject, e.g., a human patient having a CTHRC1-associated disorder or disease and/or CTHRC1-associated cancer, or at risk of having a CTHRC1-associated disorder or disease and/or CTHRC1-associated cancer, e.g., the subject can have pre-cancerous cells of a CTHRC1-associated cancer. The term “non-human animals” includes mammals and non-mammals, such as non-human primates. In an embodiment, the subject is a human. The methods and compositions described herein are suitable for treating human patients having a CTHRC1-associated disorder or disease and/or CTHRC1-associated cancer described herein. Patients having a CTHRC1-associated disorder or disease and/or CTHRC1-associated cancer described herein, include those who have developed a CTHRC1-associated disorder or disease and/or CTHRC1-associated cancer described herein, but are (at least temporarily) asymptomatic, patients who have exhibited a symptom of a CTHRC1-associated disorder or disease and/or CTHRC1-associated cancer described herein, or patients having a disorder or disease related to or associated with a CTHRC1-associated disorder or disease and/or CTHRC1-associated cancer described herein.

### Methods of Treating or Preventing Disorders or Diseases

The antibody, or antigen-binding fragment thereof described herein can be used to treat or prevent CTHRC1-associated disorder or disease and/or CTHRC1-associated cancer.

The term “CTHRC1-associated disorder or disease” as used herein, includes any disorder or disease (including proliferative disorders or diseases, e.g., cancer) that is marked, diagnosed, detected or identified by a phenotypic or genotypic aberration of CTHRC1 genetic components or expression during the course or etiology of the disease or disorder. In this regard an CTHRC1/Cthrc1 phenotypic aberration or determinant may, for example, comprise increased or decreased levels of CTHRC1 protein expression on one cell population, e.g., a cancer-associated cell population (such as cancer-associated fibroblasts population), as compared to another cell population, e.g., a normal cell population, or increased or decreased CTHRC1 protein expression on certain definable cell populations, or increased or decreased CTHRC1 protein expression at an inappropriate phase or stage of a cell lifecycle. It will be appreciated that similar expression patterns of genotypic determinants (e.g., mRNA transcription levels) of CTHRC1/Cthrc1 may also be used to classify or detect CTHRC1-associated disorder or disease and/or CTHRC1-associated cancer. An “CTHRC1-associated disorder or disease and/or CTHRC1-associated cancer,” as used herein, also includes a disorder or disease characterized by infiltration of cells expressing CTHRC1 protein, e.g., cancer-associated fibroblasts (CAFs), and activated stromal cells. In one embodiment, an CTHRC1-associated disorder or disease and/or CTHRC1-associated cancer can be colorectal cancer (CRC). In one embodiment, the CTHRC1-associated disorder or disease can be pre-cancerous cells associated with a CTHRC1-associated cancer, which would benefit from treatment with an anti-CTHRC1 antibody, or antigen-binding fragment thereof of the present invention ca

[symbol]

The term “cancer,” as used herein, is meant to refer to or describe the physiological condition in mammals that is typically characterized by unregulated cell growth. Examples of cancer include, but are not limited to, carcinoma, lymphoma, blastoma, and sarcoma. More particular examples of such cancers include, but are not limited to, breast cancer (Luminal A, TNBC, Ductal), prostate cancer, squamous cell tumors, squamous cell carcinoma (e.g., squamous cell lung cancer or squamous cell head and neck cancer), neuroendocrine tumors, urothelial cancer, vulvar cancer, mesothelioma, liver cancer, bone cancer, pancreatic cancer, skin cancer, cancer of the head or neck, lung cancer, small cell lung cancer, non-small cell lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, follicular lymphoma, diffuse large B-cell lymphoma, Burkitt's lymphoma, immunoblastic large cell lymphoma, precursor B-lymphoblastic lymphoma, mantle cell lymphoma, mycosis fungoides, anaplastic large cell lymphoma, T-cell lymphoma, and precursor T-lymphoblastic lymphoma, and any combinations of said cancers. Adrenocortical carcinoma, ladder urothelial carcinoma, cervical squamous cell carcinoma, endocervical adenocarcinoma, diffuse large B cell lymphoma, glioblastoma multiforme, brain lower grade glioma, head and neck squamous cell carcinoma, hepatocellular carcinoma, lung adenocarcinoma, large squamous cell carcinoma, cutaneous melanoma, ovarian serous cystadenocarcinoma, gastric cancer, soft tissue sarcoma, testicular germ cell cancer, thymoma, thyroid carcinoma, uterine corpus endometrial carcinoma, uterine carcinosarcoma, kidney renal clear cell carcinoma, and kidney renal papillary cell carcinoma. The present invention is also applicable to treatment of metastatic cancers.

Non-limiting examples of disorders or diseases that can be treated with the antibodies, or antigen-binding fragments thereof, include those disorders or diseases discussed below. For example, suitable disorders or diseases include, but are not limited to, a variety of cancers including, but not limited to, colorectal cancer (CRC).

In one embodiment, an anti-CTHRC1 antibody, or antigen-binding fragment thereof of the present invention can be used to treat or prevent a cancer in a subject including, but not limited to colorectal cancer, melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, a cancer cell derived from a solid cancer, or hematological cancer. In one embodiment, an anti-CTHRC1 antibody, or antigen-binding fragment thereof of the present inventions can be used to treat or prevent a cancer in a subject.

In one embodiment, the antibodies of the invention are administered to a patient having a solid tumor, including an advanced solid tumor. In one embodiment, the tumor expresses CTHRC1 or contains epithelial cells or fibroblasts expressing CTHRC1. In another embodiment, the tumor does not express CTHRC1 and/or does not contain epithelial cells or fibroblasts expressing CTHRC1. In another embodiment, administration of the antibodies of the invention to a patient upregulates an immune response in the patient. In another embodiment, administration of antibodies of the invention induce cell death of CTHRC1 expressing cells.

The term “CTHRC1 expressing cells,” as used herein, refers to cells which express CTHRC1 protein, including but not limited to epithelial cells, mesenchymal fibroblasts, myeloid cells, or lymphocytes that express CTHRC1 protein. In one embodiment, CTHRC1 expression on cells is determined using immunohistochemical staining of cell membranes, where any immunohistochemical staining above background level of a cell population indicates that the cells are CTHRC1 expressing cells. In another embodiment, CTHRC1 expressing cells, e.g., CRC cancer cells expressing CTHRC1, is identified in a patient when greater than 1%, greater than 2%, greater than 3%, greater than 4%, greater than 5%, greater than 6%, greater than 7%, greater than 8%, greater than 9%, greater than 10%, greater than 15%, greater than 20%, greater than 25%, or greater than 30%, greater than 40%, greater than 50%, greater than 60%, greater than 70%, greater than 80%, greater than 90%, or more of the cells are positive for CTHRC1 expression. In one embodiment, the CTHRC1 expressing cells in the sample are epithelial cells, mesenchymal fibroblasts, myeloid cells, or lymphocytes. In another embodiment, CTHRC1 positive expression is determined based on membrane staining as determined by, e.g., immunohistochemistry (IHC) analysis.

As used herein, the term “non-cancer sample” or “normal sample” refers to a sample from a normal tissue (e.g., a colorectal tissue sample). In some embodiments, the non-cancer sample comes from the same subject, but is from a different part of the subject than that being tested. In some embodiments, the non-cancer sample is from a tissue area surrounding or adjacent to the cancer, e.g., CRC. In some embodiments, the non-cancer sample is not from the subject being tested, but is a sample from a subject known to have, or not to have, a disorder or disease in question (for example, a particular cancer such as CRC or CTHRC1-related disorder or disorder). In some embodiments, the non-cancer sample is from the same subject, but from a point in time before the subject developed cancer. In some embodiments, the reference sample is from a benign cancer sample (for example, benign colorectal cancer sample), from the same or a different subject.

Methods for detecting expression of CTHRC1 in a tumor are known in the art. For example, immunohistochemistry (IHC) analysis was used by the inventors to show that CTHRC1 is expressed in colorectal cancer (CRC) tissue.

The terms “overexpress,” “overexpression,” or “overexpressed” interchangeably refer to a gene that is transcribed or translated at a detectably greater level, usually in a cancer cell, in comparison to a normal cell. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Thus, overexpression refers to either protein or RNA levels. Overexpression can also be by 5%, 10%, 20%, 30%, 40%, 50%, 60%, 70%, 80%, 90% or more in comparison to a normal cell or comparison cell. In certain embodiments, the anti-CTHRC1 antibodies are used to treat solid tumors likely to overexpress CTHRC1.

The term “administering” as used herein is meant to refer to the delivery of a substance (e.g., an anti-CTHRC1 antibody, or antigen-binding fragment thereof) to achieve a therapeutic objective (e.g., the treatment of an CTHRC1-associated disorder or disease and/or CTHRC1-associated cancer or the inhibition or reduction of a tumor or the treatment of an inflammatory disorder). Modes of administration may be parenteral, enteral and topical. Parenteral administration is usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Methods of administering various antibody, or antigen-binding fragment thereof are known in the art and are described below. Suitable dosages of the antibody, or antigen-binding fragment thereof used will depend on the age and weight of the subject and the particular drug used.

In an embodiment, the antibody, or antigen-binding fragment thereof is administered to the subject (e.g., a human subject) intravenously. In an embodiment, the antibody, or antigen-binding fragment thereof is administered to the subject at a dose between 0.1 mg/kg and 50 mg/kg, e.g., between 0.2 mg/kg and 25 mg/kg, or between 1 mg/kg and 5 mg/kg. In an embodiment, the antibody, or antigen-binding fragment thereof is administered to the subject at a fixed dose between 10 mg and 1000 mg, e.g., between 10 mg and 500 mg, between 10 mg and 250 mg, between 50 mg and 100 mg, between 100 mg and 150 mg. between 100 mg and 200 mg, or between 150 mg and 250 mg. In an embodiment, the antibody, or antigen-binding fragment thereof is administered once a week, twice a week, once every two weeks, once every three weeks, once every four weeks, once every eight weeks, once a month, once every two months, or once every three months.

The antibody, or antigen-binding fragment thereof can be used by themselves or conjugated to a second agent, e.g., a bacterial agent, toxin, or protein, e.g., a second anti-CTHRC1 antibody, or antigen-binding fragment thereof. This method includes: administering the antibody, or antigen-binding fragment thereof, alone or conjugated to a second agent, to a subject requiring such treatment. The antibody, or antigen-binding fragment thereof can be used to deliver a variety of therapeutic agents, e.g., a toxin, or mixtures thereof.

### Combination Therapies

The antibody, or antigen-binding fragment thereof can be used in combination with other therapies. For example, the combination therapy can include an antibody, or antigen-binding fragment thereof co-formulated with, and/or co-administered with, one or more additional therapeutic agents, e.g., one or more additional therapeutic agents described herein. In other embodiments, the antibody, or antigen-binding fragment thereof is administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder or disease. In an embodiment, two or more treatments are delivered prophylactically, e.g., before the subject has the disorder or disease or is diagnosed with the disorder or disease. In another embodiment, the two or more treatments are delivered after the subject has developed or diagnosed with the disorder or disease. In an embodiment, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In an embodiment of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In an embodiment, delivery is such that the reduction in a symptom, or other parameter related to the disorder or disease is greater than what would be observed with one treatment delivered in the absence of the other. The effect of the two treatments can be partially additive, wholly additive, or greater than additive. The delivery can be such that an effect of the first treatment delivered is still detectable when the second is delivered.

In an embodiment, the additional agent is a second antibody, or antigen-binding fragment thereof, e.g., an antibody, or antigen-binding fragment thereof different from a first antibody, or antigen-binding fragment thereof. Exemplary antibody, or antigen-binding fragment thereof that can be used in combination include, but are not limited to, any combination of the antibody, or antigen-binding fragment thereof listed in Tables 1-2.

In an embodiment, the antibody, or antigen-binding fragment thereof is administered in combination with a second therapy to treat or prevent a CTHRC1-associated disorder or disease and/or CTHRC1-associated cancer described herein.

Exemplary therapies that can be used in combination with an antibody, or antigen-binding fragment thereof or composition described herein to treat or prevent other disorders or diseases are also described in the section of “Methods of Treating or Preventing Disorders or Diseases” herein.

In one embodiment, the anti-CTHRC1 antibody, or antigen-binding fragment thereof of the invention is administered in combination with one or more immune checkpoint inhibitors (e.g., antibody or small molecule immune checkpoint inhibitors) for the treatment of a cancer. In some embodiments, the immune checkpoint inhibitor is an inhibitor (e.g., an antibody) of Programmed Death-Ligand 1 (PD-L1, also known as B7-H1, CD274), Programmed Death 1 (PD-1), CTLA-4, PD-L2 (B7-DC, CD273), LAG3, TIM3, 2B4, 4-1BB, A2aR, B7H1, B7H3, B7H4, BTLA, CD2, CD6 (itolizumab), CD27, CD28, CD30, CD38, CD39, CD40, CD47, CD70, CD73, CD80, CD86, CD166, CD137, CD160, CD166, CD200, CD200R1, CD226, CD276, CSF1R, DR3, GAL9, G1TR, HVEM, IDO1, IDO2, ICOS (inducible T cell costimulator), PSGL1, CLEC4A, KIR, LAG3, LAIR1, TREM2, LILRB1, LILRB2, LILRB3, LIGHT, MARCO (macrophage receptor with collageneous structure), PS (phosphatidylserine), OX-40, SLAM, SIRPA, CSF1R, CD47, SIRPA, TIGHT, TIM3, TGFβ, VISTA, VTCN1, or any combinations thereof.

In some embodiments, the immune checkpoint inhibitor is an anti-CTLA-4, PD-L1, or PD-1 antibody therapy such as, but not limited to Yervoy® (ipilimumab; Bristol-Myers Squibb), Opdivo® (nivolumab; Bristol-Myers Squibb), Keytrada® (pembrolizumab; Merck), and Tecentriq® (atezolizumab; Roche).

In other embodiments, the immune checkpoint inhibitor is an anti-CD38 antibody therapy such as isatuximab (Sanofi), Darzalex® (daratumumab; Genmab A/S and Janssen Biotech), MOR202 (MorphoSys AG), and Tusk Therapeutics Ltd.'s anti-CD38 monoclonal antibody.

In one embodiment, the anti-CTHRC1 antibody, or antigen-binding fragment thereof of the invention is administered in combination with one checkpoint inhibitor, e.g., an anti-CTLA-4, CD38, PD-L1, or PD-1 antibody. In other embodiments, the anti-CTHRC1 antibody, or antigen-binding fragment thereof of the invention is administered in combination with more than one checkpoint inhibitor, e.g., an anti-CTHRC1 antibody, or antigen-binding fragment thereof of the invention in combination with an anti-CD38 antibody and an anti-PD-L1 antibody, or an anti-CTHRC1 antibody, or antigen-binding fragment thereof of the invention in combination with an anti-CD38 antibody and an anti-PD-1 antibody.

In some embodiments, the checkpoint inhibitor is an antibody currently undergoing clinical testing, including, for example, IDO (Epacadostat and Indoximod and BMS-986205), 4-1BB/CD137 (Utomilumab and Urelumab), KIR (Lirilulmab), CD40 (CP-870,893), CD27 (Varlilumab), LAG-3 (Relatilimab), MHCII (Eftilagimod Alpha).

In some cases, the immune checkpoint inhibitor (e.g., anti-CTLA-4 antibody, an anti-PD-1 antibody, an anti-PDL-1 antibody, Lag3, LAIR1, or LAIR 2) is administered at a dosage of 0.01-10 mg/kg (e.g., 0.01, 0.05, 0.1, 0.5, 1, 5, or 10 mg/kg) bodyweight. For example, the PDL-1 inhibitor is administered in an amount of 0.01-30 mg (e.g., 0.01, 0.05, 0.1, 0.5, 1, 5, 10, 20, or 30 mg) per dose. In another example, the antibody is administered in the dose range of 0.1 mg/kg to 10 mg/kg of body weight. In some cases, the anti-CTHRC1 antibody is administered in an amount of 0.01-30 mg (e.g., 0.01, 0.05, 0.1, 0.5, 1, 5, 10, 20, or 30 mg) per dose.

Drug therapy may be used alone, or in combination with other treatments such as surgery or radiation therapy. Several classes of drugs may be used in cancer treatment, depending on the nature of the organ involved. For example, breast cancers are commonly stimulated by estrogens, and may be treated with drugs which inactive the sex hormones. Similarly, prostate cancer may be treated with drugs that inactivate androgens, the male sex hormone.

In particular embodiments, the anti-CTHRC1 antibody, or antigen-binding fragment thereof can be administered alone or with another anti-cancer agent which acts in conjunction with or synergistically with the antibody to treat the disease or disorder associated with CTHRC1 activity. Such anti-cancer agents include, for example, agents well known in the art (e.g., cytotoxins, chemotherapeutic agents, small molecules and radiation). Examples of anti-cancer agents include, but are not limited to, Panorex (Glaxo-Welcome), Rituxan (IDEC/Genentech/Hoffman la Roche), Mylotarg (Wyeth), Campath (Millennium), Zevalin (IDEC and Schering AG), Bexxar (Corixa/GSK), Erbitux (Imclone/BMS), Avastin (Genentech) and Herceptin (Genentech/Hoffman la Roche). Other anti-cancer agents include, but are not limited to, those disclosed in U.S. Pat. No. 7,598,028 and International Publication No. WO2008/100624, the contents of which are hereby incorporated by reference. One or more anti-cancer agents may be administered either simultaneously or before or after administration of an antibody or antigen-binding fragment thereof.

In particular embodiments of the invention, the anti-CTHRC1 antibody, or antigen-binding fragment thereof described herein can be used in a combination therapy with an inhibitor of NAMPT (see examples of inhibitors in US 2013/0303509; Abb Vie, Inc., incorporated by reference herein) to treat a subject in need thereof. NAMPT (also known as pre-B-cell-colony-enhancing factor (PBEF) and visfatin) is an enzyme that catalyzes the phosphoribosylation of nicotinamide and is the rate-limiting enzyme in one of two pathways that salvage NAD. In one embodiment, anti-CTHRC1 antibodies described herein are administered in combination with a NAMPT inhibitor for the treatment of cancer in a subject.

### Methods of Diagnosis

In some aspects, the present disclosure provides a diagnostic method for detecting the presence of CTHRC1 (e.g., human CTHRC1) in vitro (e.g., in a biological sample, such as a biopsy or blood sample) or in vivo (e.g., in vivo imaging in a subject). The method includes: (i) contacting the sample with an antibody, or antigen-binding fragment thereof described herein, or administering to the subject, the antibody, or antigen-binding fragment thereof; (optionally) (ii) contacting a reference sample, e.g., a control sample (e.g., a control biological sample, such as a biopsy or blood sample) or a control subject with an antibody, or antigen-binding fragment thereof described herein; and (iii) detecting formation of a complex between the antibody, or antigen-binding fragment thereof and CTHRC1 in the sample or subject, or the control sample or subject, wherein a change, e.g., a statistically significant change, in the formation of the complex in the sample or subject relative to the control sample or subject is indicative of the presence of CTHRC1 in the sample. The antibody, or antigen-binding fragment thereof can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting a polypeptide (e.g., CTHRC1) or a nucleic acid encoding the polypeptide includes, but is not limited to, cells, cell lysates, proteins or membrane extracts of cells, body fluids such as blood, or tissue samples such as biopsies.

Complex formation between the antibody, or antigen-binding fragment thereof, and CTHRC1, can be detected by measuring or visualizing either the antibody, or antigen-binding fragment thereof bound to CTHRC1 or unbound antibody, or antigen-binding fragment thereof. Any suitable detection assays can be used, and conventional detection assays include an enzyme-linked immunosorbent assays (ELISA), a radioimmunoassay (RIA) or tissue immunohistochemistry. Alternative to labeling the antibody, or antigen-binding fragment thereof, the presence of CTHRC1 can be assayed in a sample by a competition immunoassay utilizing standards labeled with a detectable substance and an unlabeled antibody, or antigen-binding fragment thereof. In this assay, the biological sample, the labeled standards and the antibody, or antigen-binding fragment thereof are combined and the amount of labeled standard bound to the unlabeled binding molecule is determined. The amount of CTHRC1 in the sample is inversely proportional to the amount of labeled standard bound to the antibody, or antigen-binding fragment thereof.

The antibody, or antigen-binding fragment thereof described herein can be used to diagnose disorders or diseases that can be treated or prevented by the antibody, or antigen-binding fragment thereof described herein. The detection or diagnostic methods described herein can be used in combination with other methods described herein to treat or prevent a disorder or disease described herein.

### EXAMPLES

### Example 1: Explore CTHRC1 Function in CRC Tumor Development

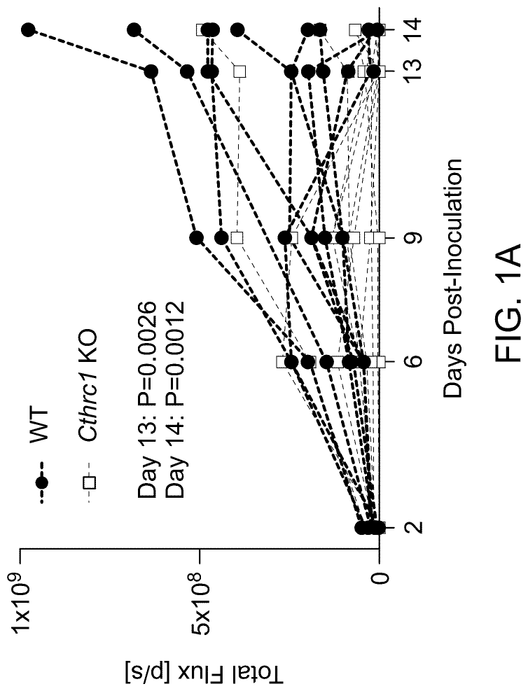

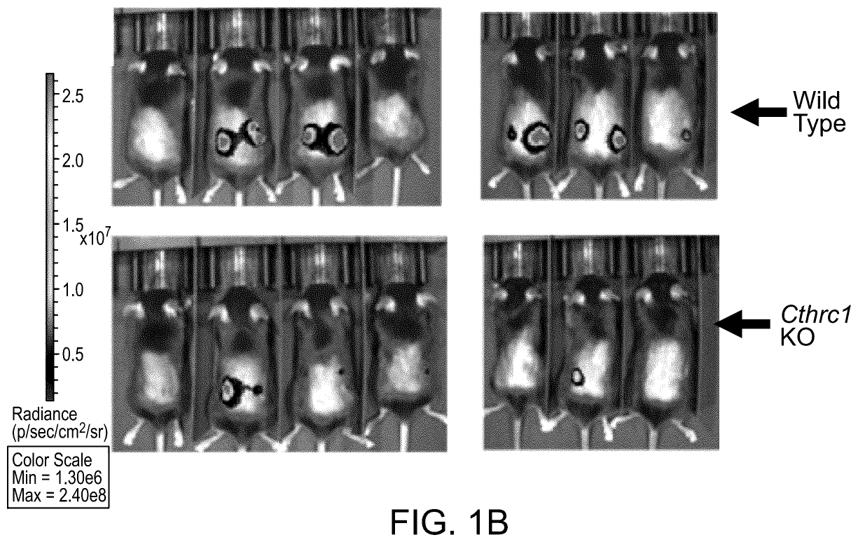

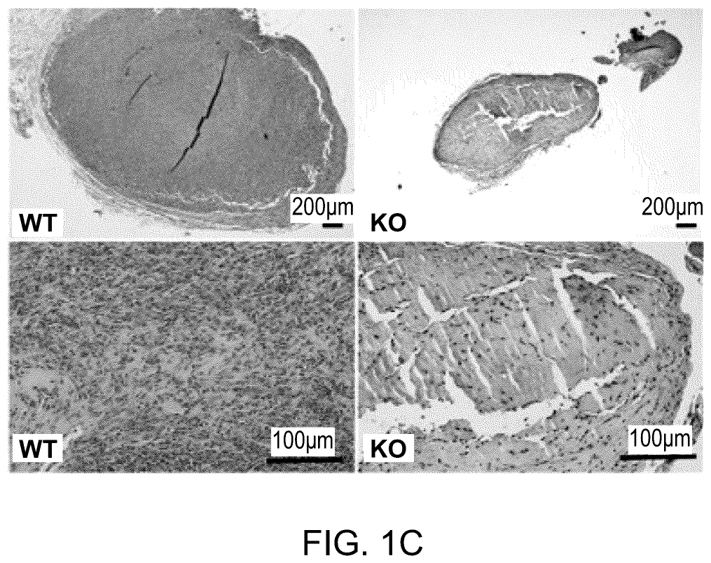

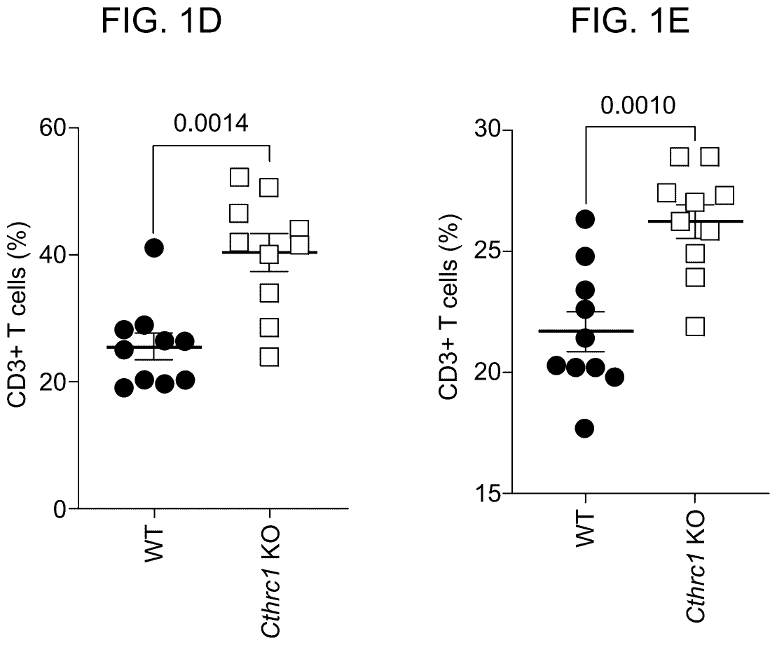

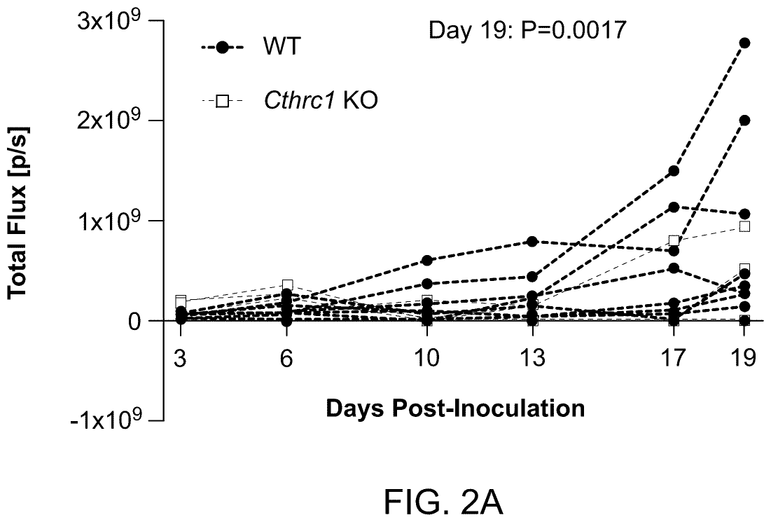

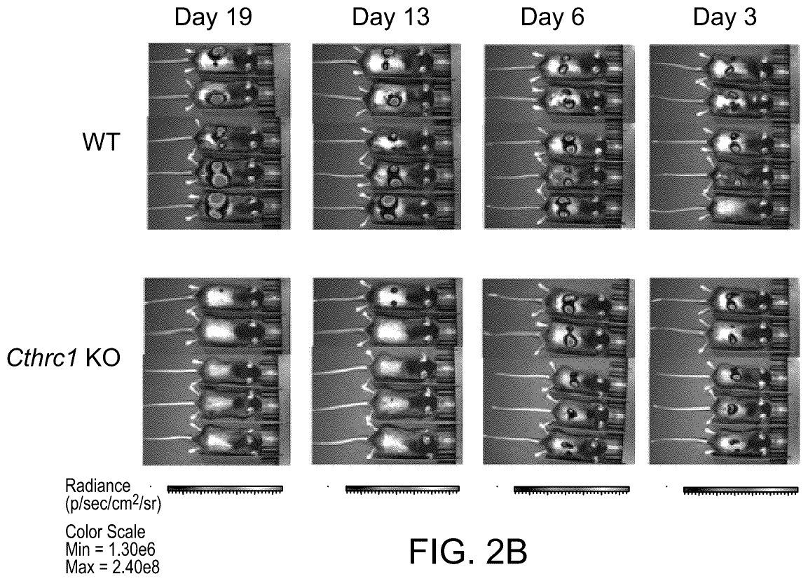

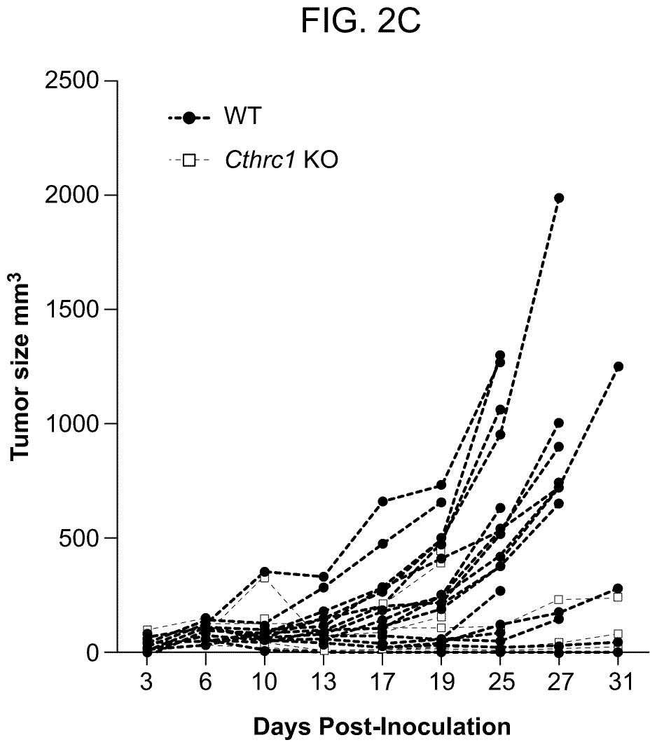

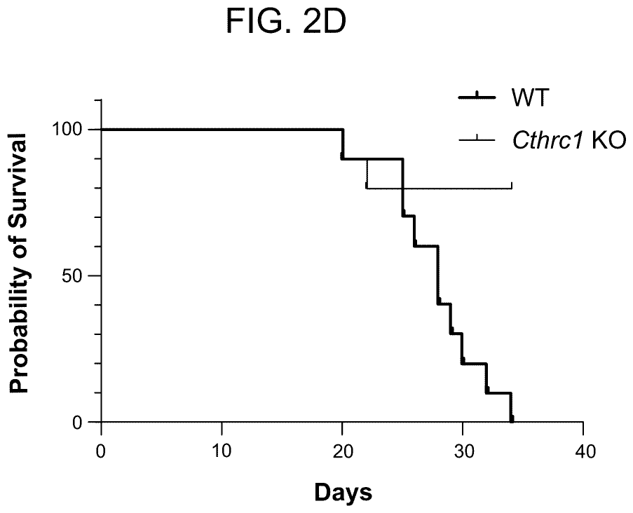

The results of the initial experiment demonstrated that MC38Luc+/RFP+ CRC cells grow significantly slower in Cthrc1 global KO mice (C57BL/6J-Cthrc1^{tm1Vli}) than in C57BL/6J wild type (WT) mice in a subcutaneous model (FIGS. 1A-1E and 2A-2D). In this experiment, tumor burden was measured by BLI twice weekly and by tumor mass upon euthanasia. Mice were inoculated on Day 0 and euthanized on Day 16, at which point tumors and spleens were removed for histology and flow cytometry immune cell analysis. The data analyses revealed that knocking out Cthrc1 in mice significantly decreased tumor burden (FIGS. 1A and 1B), altered tumor morphology and cellular content (FIG. 1C), and increased T-cell numbers in tumors (FIG. 1D) and the spleen (FIG. 1E). In the absence of immunosuppressive effects of CTHRC1, increase of CD3+ cells in the tumor and spleen led us to hypothesize that Cthrc1 regulates immunosuppressive actions of myeloid cells, or has direct effects on T-cells, which will be explored in this pilot.

### Example 2: Cthrc1 KO Mice with MC38 Tumors Exhibit Longer Survival Compared to WT Mice

In a second study, subcutaneous MC38 tumors grown in WT and Cthrc1 KO mice were monitored with BLI (FIGS. 2A and 2B) and caliper measurements (FIG. 2C). Results from this study validated the BLI and caliper data in the first study, and Kaplan-Meier survival analysis showed significantly increased survival of Cthrc1 KO vs WT mice (FIG. 2D). Mice were euthanized when their tumors begin to ulcerate, or reached a maximum size allowed by our IACUC protocol. The data determined CTHRC1 to be critical for the survival of a colorectal cancer (CRC) cell line, MC38, supporting the potential for CTHRC1-neutralizing antibodies in oncologic applications.

### Example 3: CTHRC1-Neutralizing Abs Induce Tumor Regression in a Similar Manner to Cthrc1 KO Mice

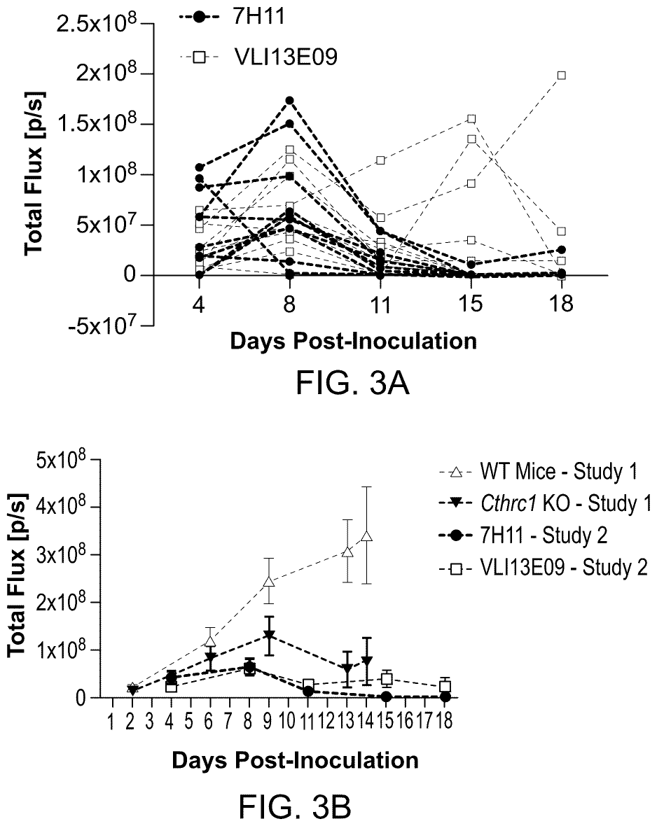

The third experiment aimed to test if the previously-generated CTHRC1-neutralizing antibodies recapitulate CTHRC1-deficiency effects on tumor growth in vivo. Two CTHRC1-neutralizing Abs (VLI13E09 and 7H11) were tested in C57BL/6J mice over the course of 18 days with four days of treatment interspersed (FIGS. 3A and 3B). This study revealed a similar pattern of tumor regression seen in both previous Cthrc1 KO studies (FIG. 3A). When overlayed with the data from our first, predetermined-endpoint Cthrc1 KO study, it is found that tumor growth was similarly attenuated (FIG. 3B). Mice tolerated the treatments well.

### Example 4: CTHRC1 Induces Immunosuppressive Myeloid Cell Differentiation

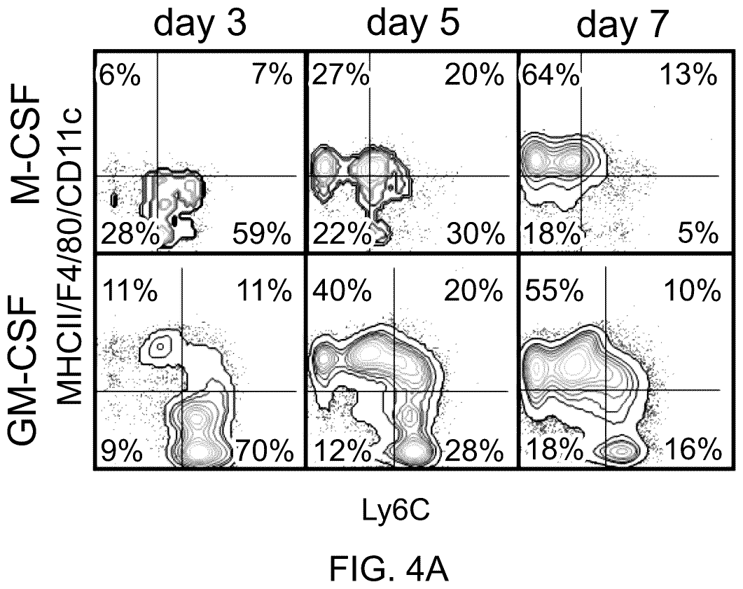

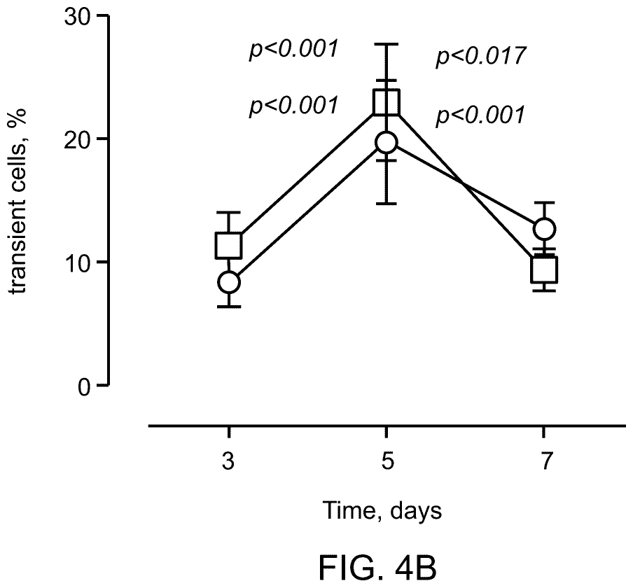

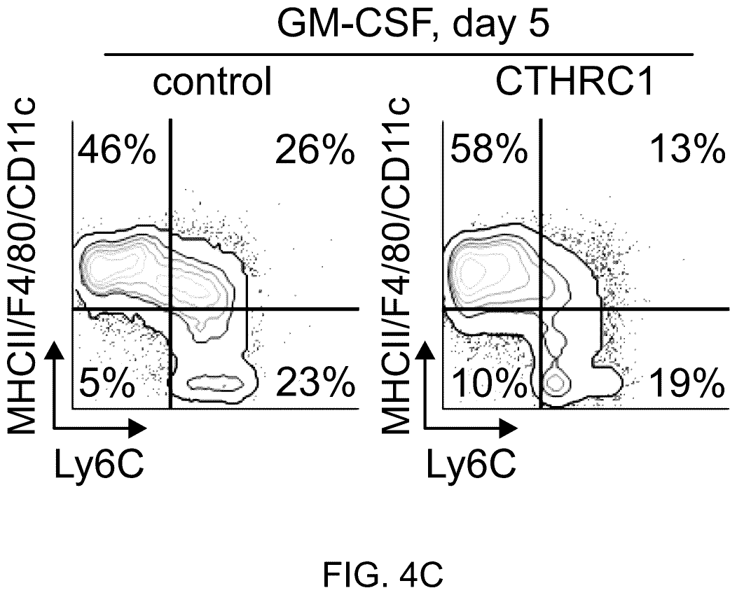

Myeloid cells differentiate from monocytes/myeloid progenitors (Ly6C+ cells, such as MDSCs) into mature myeloid cells (MHCII+, F4/80+, and/or CD11c+, and Ly6C−, such as TAMs). This differentiation is induced by granulocyte-macrophage colony-stimulating factor (GM-CSF), commonly found within the tumor setting, and by macrophage-CSF (M-CSF). As cells transition from the immature myeloid into the mature myeloid cell state, they often express both marker profiles, and thus, these cells were termed transient or transitioning cells. Mature myeloid cells, such as M2 polarized TAMs, are strongly immunosuppressive; they inhibit the proliferation and activation of T-cells, which allows tumor cells to evade the immune system and proliferate. In vitro experiment was performed with C57BL/6J mouse bone marrow Lin-negative hematopoietic progenitor cells (HPCs) to explore the effect of recombinant CTHRC1 on this process. In vitro flow cytometry data demonstrated that treatment of murine myeloid cells with M-CSF or GM-CSF induced myeloid cell maturation (FIG. 4A), and that the largest number of cells in the transitioning stage occurs at day 5 (FIG. 4B). Importantly, when developed recombinant CTHRC1 was added to these cultures (at day 2 and 4), there were less immature and transitioning cells, and more mature cells, compared to vehicle control levels (FIG. 4C). These data further supports our hypothesis that CTHRC1 induces immunosuppressive myeloid cell differentiation, which could explain the increases in T cells in the Cthrc1 KO mice (FIGS. 1D and 1E) and decreased tumor growth in these mice, as well as mice treated with CTHRC1 Abs (FIG. 3). It is thus critical to find effective ways to reverse immunosuppression to stimulate the adaptive immune system's tumor-killing effects.

### Example 5: Determine the Efficacy of CTHRC1 Neutralizing Antibodies at Reducing CRC Burden In Vivo

Two separate neutralizing antibodies (Abs) against CTHRC1 (Vli13E09 and 7H11) and one control Ab will be tested and compared, to mice with no treatment, in our C57BL/6J-subcutaneous colorectal tumor (MC38^{Luc+/RFP+}) syngeneic mouse model using bioluminescent imaging (BLI) and caliper measurements for tumor growth. Spleens and tumors will be weighed and used for immune cell analyses described in Example 6. Blood serum will be used to determine levels of circulating CTHRC1. Tumor tissue sections will be used for IHC analyses. It is expected that both CTHRC1 Abs will decrease tumor growth compared to control groups.

C57BL/6 mice (~12 weeks old, males and females, n=10/group, per sex) will be subcutaneously injected with 5×105 MC38Luc+/RFP+ cells in Matrigel on both flanks, and tumor growth will be quantified with BLI in vivo bi-weekly as was done in our three preliminary data studies. At ~1-week after inoculation, mice will be randomized into 4 groups based on tumor burden (BLI). Groups will be: 1) No treatment; 2) Control Ab 3) CTHRC1-neutralizing Ab, Vli13E09 (also referred to as 13E09), which was developed and described and; 4) CTHRC1-neutralizing Ab, 7H11. Both 13E09 and 7H11 antibodies were raised against full length human CTHRC1 which was expressed in E. coli. The control Ab will be an IgG1kappa raised against human serum albumin from BBI™ Solutions antibody development pipeline in Portland, Maine. Mice will be treated with CTHRC1 antibodies or control antibodies (20 mg/kg), or nothing, via intraperitoneal injections twice weekly. Mice will be weighed and checked regularly. All the mice will be euthanized at the same time (within 1 day of each other) when the control group begins to reach the tumor size maximum allowed by our IACUC protocol (e.g., when 1 or more mice meet either of the following criteria: ulcerating tumor, tumor >2 cm along any axis, or tumor mass >10% mouse mass). All mice will then be anesthetized prior to retro-orbital blood exsanguination and euthanasia. Spleens and tumors will be weighed and used for analysis (see Example 6) and blood serum will be stored and assessed for CTHRC1 via ELISA. Tumor and spleen tissue samples will also be used for IHC analyses of tumor cells, and surrounding and infiltrating stroma and immune cells. From our preliminary study, n=10 mice per group should be appropriate to detect effects of CTHRC1 on colorectal cancer tumor growth based on BLI and tumor mass at the endpoint. Performing the work in male and female mice will be able to determine if the results are sexually-dimorphic.

### Example 6: Determine Mechanisms by which CTHRC1 Inhibition Affects Tumor Cells and Immune Surveillance

The mechanisms by which inhibition of our target of interest affects tumor cells and immune surveillance will be determined. Tumor and host cell changes in tissues from Example 5 (spleens and tumors) will be explored by flow cytometry and single cell RNASeq (scRNASeq) to test the hypothesis that CTHRC1 reduces immunosurveillance by promoting the differentiation of immunosuppressive MDSCs and TAMs. Single cell RNA sequencing will be performed on all cells in the tumors (immune, stroma, and tumor cells) isolated from all groups of mice to test the hypothesis that therapy-treated tumors will be less immunosuppressed and smaller versus controls. Analyses will be performed to help determine what specific immune cells and pathways within the host and the tumor cells (e.g. apoptosis, cell cycle arrest, etc.) could explain the expected reduced primary tumor burden. It is hypothesized that CTHRC1-Ab treated tumors will be more apoptotic, or less proliferative, and be substantially less immunosuppressed than controls. Analysis will be done to help determine what specific immune cells and pathways within the host and the tumor cells (e.g. apoptosis, cell cycle arrest, etc.) could explain the reduced primary tumor burden expected to be observed.

Next the mechanisms by which CTHRC1 inhibition affects CRC cell and immune cell transcriptomic profiles (scRNAseq), and immune states and potential for surveillance (scRNAseq and flow cytometric immune profiling) will be explored. Assessment the hematopoietic cell compositions of tumors and spleens of mice in Example 5 will be performed using flow cytometry methods established by collaborators to perform and analyze single cell RNA sequencing data of tumors from at least 3 mice/group. This will allow for evaluating cellular identities and molecularly-distinct immune sub-populations based on transcriptome differences within tumors using an unbiased manner with established pipelines commonly used by the core. Tissues will be fixed and processed for scRNAseq. Overall, this work will test the hypothesis that CTHRC1 promotes tumor progression via differentiation of immunosuppressive TAMs that facilitate tumor immune escape. A Naïve Bayes classifier using the R package caret will be trained and used to compare immune and stromal compositions in tumors from different treatment groups. If CTHRC1-Ab treated mice have a higher TAM/lower T cell phenotypes than the Control-Ab treated mice and mice with no treatment, the results with IHC staining or with immunofluorescence can be confirmed.

### Example 7: 2^{nd} Cthrc1 KO Study In Vivo

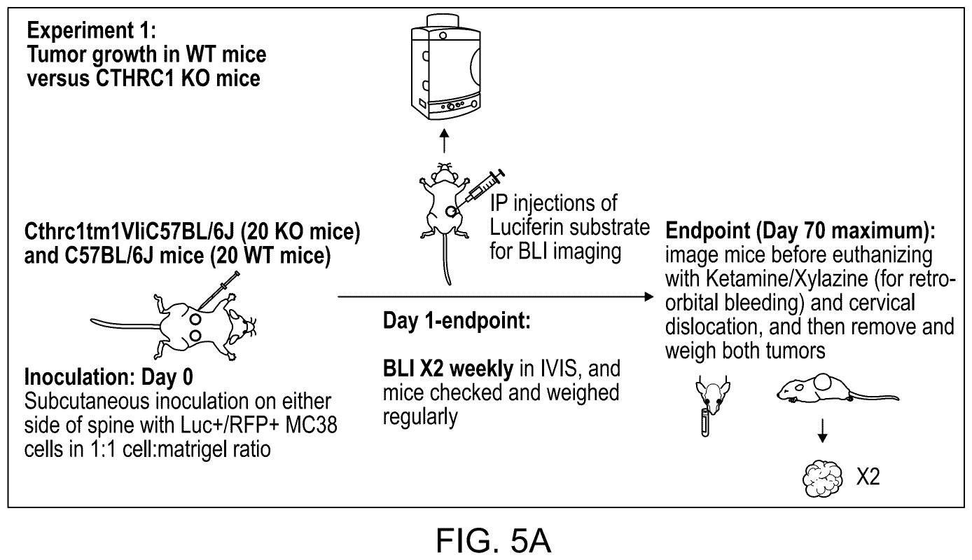

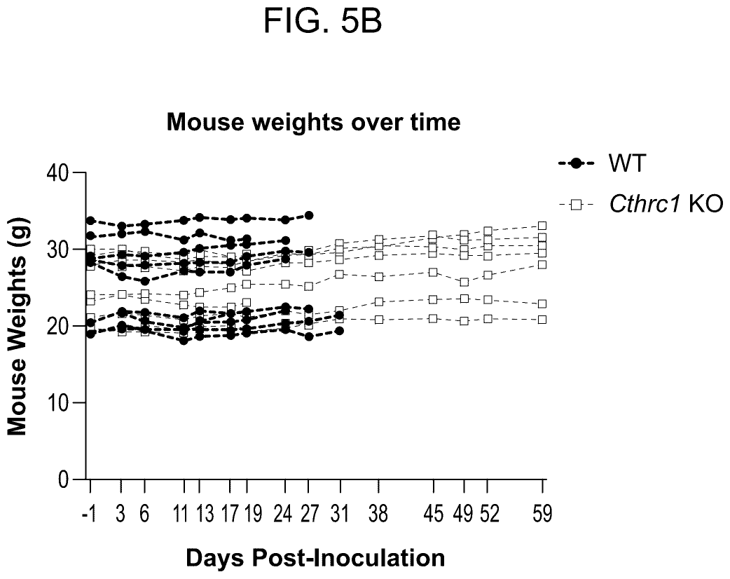

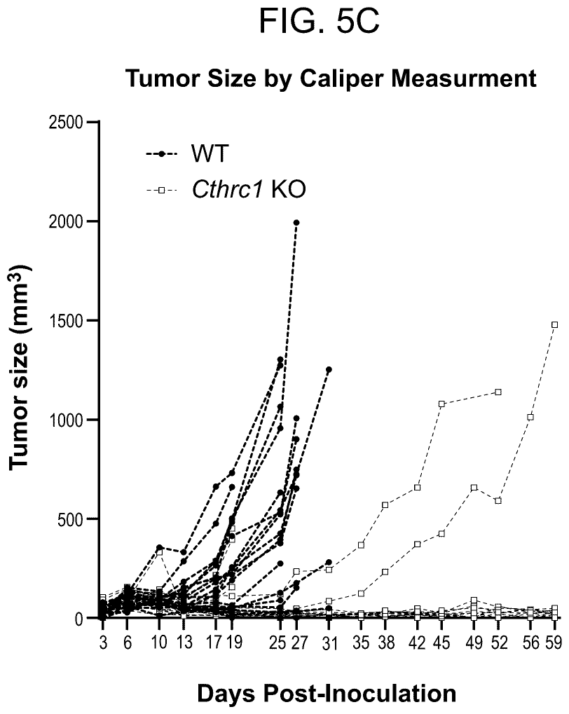

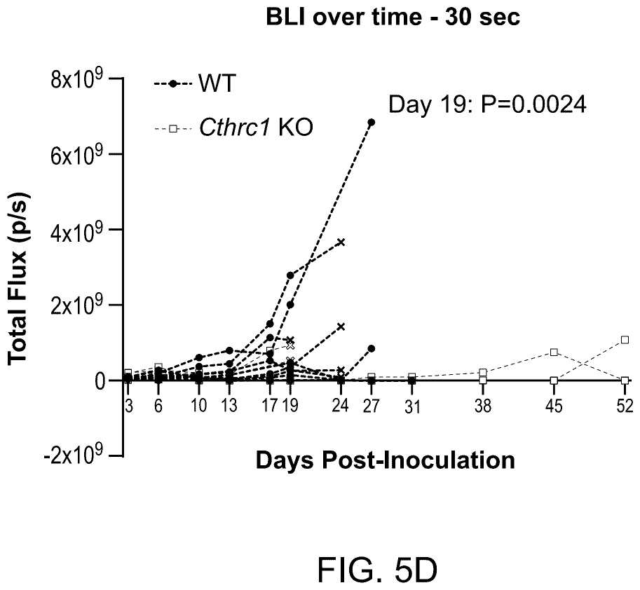

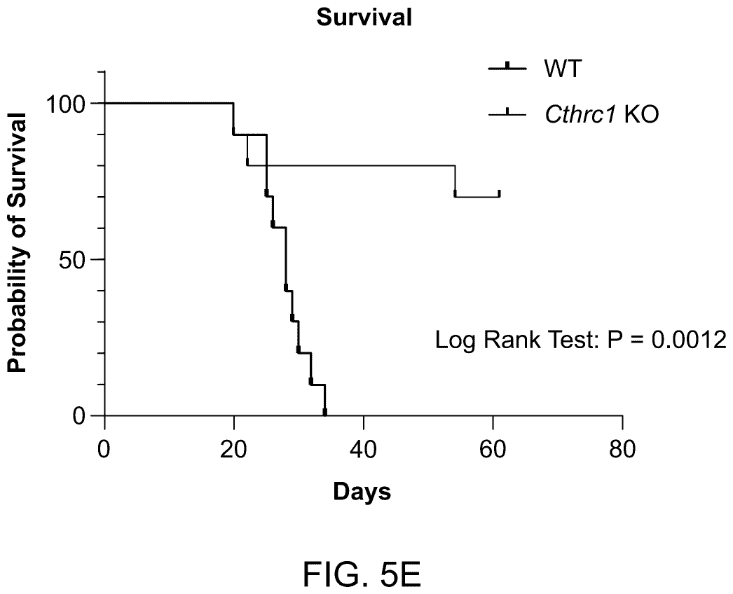

Another study of colorectal cancer growth controls (C57BL/6J) mice or Cthrc1-knocked out (global knockout mice) mice was performed. Results are shown here in FIGS. 5A-5D. Growth of tumor on the backs of mice were measured with calipers, and bioluminescence.

### Example 8: Identification of T Cell Immunoglobulin and Mucin Domain Containing 4 (TIMD4) as a Receptor for CTHRC1 Mediating T Cell Proliferation

CTHRC1 is a secreted protein produced transiently by activated fibroblasts during tissue repair. In addition to its inducible expression in response to tissue injury, CTHRC1 is also constitutively expressed in bone by osteocytes and osteoblasts, which accounts for levels observed in circulation (Toomey et al., Vascul Pharmacol. 2023 December; 153:107246. doi: 10.1016/j.vph.2023.107246. Epub 2023 Nov. 29.PMID: 38040222). As a secreted protein with effects on colorectal tumor growth it is hypothesized that CTHRC1 mediates its effects on cells after binding to a cell surface receptor with downstream activation of signaling pathways. Although various proteins have been claimed to function as CTHRC1 receptors (Yamamoto et al. Dev Cell. 2008 July; 15 (1): 23-36. doi: 10.1016/j.devcel.2008.05.007.PMID: 18606138; Balazova et al, Nat Commun. 2021 Dec. 8; 12 (1): 7144.doi: 10.1038/s41467-021-27442-x.), convincing evidence of CTHRC1 binding to a cell surface receptor has been lacking. Previous studies indicated that CTHRC1 has immunosuppressive effects in a model of inflammatory arthritis (Jin et al, Bone. 2017 April; 97:153-167. doi: 10.1016/j.bone.2017.01.022. Epub 2017 Jan. 21.) and in tumors, suggesting that immune cells might be a target for CTHRC1.

### Methods and Results

Cell isolation: Based on published data (Jin et al. Bone (2017)) of Cthrc1 null mice suffering from massive inflammation with bone and cartilage destruction in a model of inflammatory arthritis, it is hypothesized that immune cells are a target for CTHRC1. To identify putative cell surface binding proteins for CTHRC1, cells from spleens were harvested from Cthrc1 null mice (Cthrc1^{tm1Vli}). Spleens were minced in 10 ml PBS/EDTA (2 mM) and pushed through a 100 μm cell strainer, followed by pipetting through a 40 μm cell strainer into a tube containing 10 ml PBS/EDTA. Cell strainers were washed with an additional 12 ml of PBS/EDTA. Cells were spun at 350×g for 3 minutes and resuspended in 1 ml ACK lysis buffer ThermoFisher #A1049201) for 5 minutes at room temperature. 10 ml of isolation buffer (IB: PBS, 2 mM EDTA, 0.1% BSA) was added, and cells were spun and used for experiments.

CTHRC1 production: CTHRC1 containing conditioned media were prepared by transfecting HEK293T cells (ATCC) growing in 10 cm tissue culture dishes. For production of active CTHRC1, a pTT5 expression vector was designed encoding the N terminally truncated VVD-CTHRC1 (VVDCM, amino acids 47-243 of human CTHRC1) following cleavage of the N-terminal Ig kappa signal peptide derived from the pDisplay vector (Life Technologies, Toomey et al PMID: 38040222). Control conditioned media (CtrlCM) were generated by transfection of HEK293T cells with empty pTT5 vector. X-tremeGENE™ 9 DNA transfection reagent was used following the manufacturer's instructions (Sigma-Aldrich).

One day after transfection, media were replaced with serum free (SF) DMEM (Dulbecco's Modification of Eagles' Medium containing 3.7 g/L NaHCO₃, pH7.4; Corning Cat. #50-003-PB) and incubated for an additional 48 hours. Media were then collected, cells were removed by centrifugation, media were filtered through a 0.20 μm filter, frozen on dry ice and stored at −20° C. until use. VVD-CTHRC1 levels were measured in all VVDCM preparations as previously described (Toomey et al, 2023) and were found to be approximately 100 ng/ml.

CTHRC1 binding to cells: Anti-CTHRC1 antibody clone Vli13E09 (mhir.org/center-for-molecular-medicine/shared-research-facilities/antibody-products/, Toomey et al PMID: 38040222) was conjugated to Alexa-Fluor 488 (ThermoFisher, #A20000) following the protocol provided by the manufacturer. Approximately 5×10⁷ cells isolated from spleens of Cthrc1 null mice were incubated in 15 ml of VVDCM for 1 hour at 4° C. Cells were then washed with FACS buffer (PBS, 2 mM EDTA, 0.5% BSA), followed by incubation with anti-CTHRC1 Vli13E09-Alexa 488 (100 ng/ml) for >45 minutes at 4° C. Two 1 ml aliquots of cells were removed, each washed with 10 ml of cold FACS buffer, cells from both tubes were resuspended in 1 ml FACS buffer and strained through a 40 μm cell strainer into a tube with 1 ml 2×DAPI solution (BD Biosciences #564907). Cells were then sorted on a FACSAria cell sorter into two fractions: one that was highly labeled with anti-CTHRC1 Vli13E09-Alexa488 (labeled cells), and a second fraction of cells with minimal to no labeling with the antibody (unlabeled). After sorting, cells from each sample were spun and resuspended in 200 μl of RLT buffer (Qiagen) with 1 μl RNAse inhibitor (New England Biolabs, #M0314) for RNA isolation, followed by freezing isolated RNA on dry ice. RNA from labeled und unlabeled cells from four separate spleens was analyzed by RNA-seq.

### RNA-Seq Analysis:

Cell pellets were snapfrozen and stored at −80 C.° prior to processing using the Qiagen RNeasy Plus Mini kit. RNA was quantified by qubit and integrity measured on a Tape Station instrument (Agilent). For RNA-seq, 100 ng RNA was used as input into the Quantseq FWD workflow (Lexogen) for library preparation following manufacturer's instructions. Libraries were pooled for sequencing on an Illumina NextSeq2000 instrument targeting 10 M, single-end 100 bp reads per sample. Differential gene expression was performed using DESeq2 (v1.46.0; Love et al., 2014) within the nf-core/rnaseq workflow, applying shrinkage estimation for dispersion and log 2 fold changes using apeglm (v1.28.0) for adaptive t prior shrinkage estimation (Zhu, Ibrahim, and Love 2018). An adjusted p-value (FDR) threshold of 0.1 was used to define statistical significance.

The analysis focused on genes with a transmembrane domain highly enriched in the samples of cells labeled with Vli13E09-Alexa488. One of those sequences was TIMD4 (Uniprot ID Q6U7R4) with 20.5-fold higher expression levels (adjusted p=0.000835) in the labeled cell population. Another cell surface receptor with 37-fold higher expression levels in VVD-CTHRC1 binding cells was PAQR9 (adjusted p=0.006).

### Visualization of VVD-CTHRC1 Binding to TIMD4:

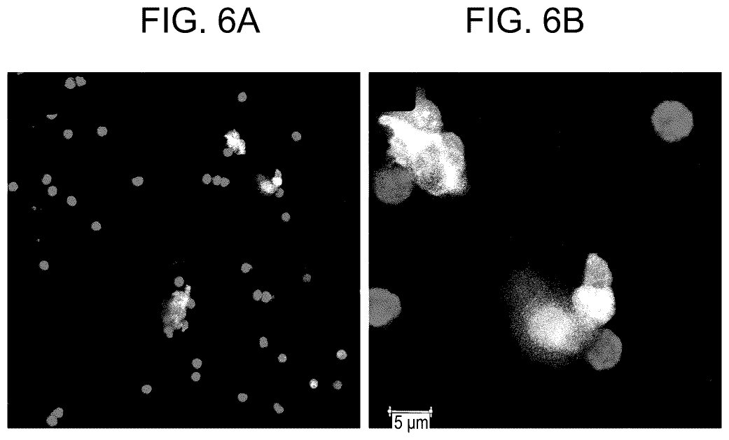

In two experiments before cells were sorted, a sample of cells was viewed by confocal microscopy. 5 ml of cells were rinsed with FACS buffer, fixed in 1 ml Formalin (10%) for 5 minutes, washed with 1 ml PBS, and resuspended in 0.1 ml PBS. Cells were dropped onto a glass slide, partially air dried, further adhered to the slide with a drop of Formalin for 5 minutes, and cells were washed with water, stained with Hoechst 33342 (1 μg/ml; Sigma), washed with water, and mounted in under a coverslip. Cells were viewed by confocal microscopy (Leica). FIGS. 6A-6B shows binding of CTHRC1 (in green) to a population of cells, approximately 2.5%-7% of total cells isolated, depending on the spleen analyzed. Additional characterization of cells binding VVD-CTHRC1 by flow cytometry using cell surface markers identified these cells as T-cells (CD3+, data not shown).

An expression plasmid encoding human TIMD4 (TIM4) with a C terminal Myc-DDK tag was obtained from OriGene Technologies Inc. (#RC203848, NM_138379). Another expression plasmid encoding the cell surface receptor murine PAQR9 with a C terminal Myc-DDK tag was also obtained from the same vendor (#MR215906, NM_198414). Transfection of CHO-K1 cells with these plasmids was performed followed by immunofluorescence confocal microscopy. For this purpose, CHO-K1 cells were grown on fibronectin coated coverslips in DMEM containing 10% fetal bovine serum. X-tremeGENE™ 9 DNA transfection reagent was used and cells were grown for 48 hours at 37° C. in a CO₂ (5%) incubator.

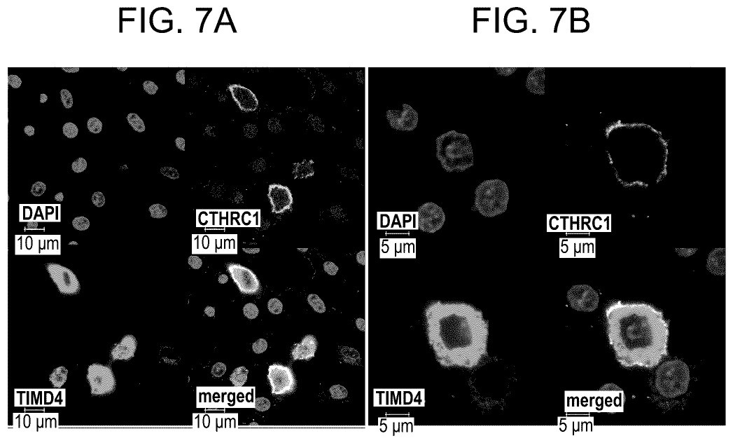

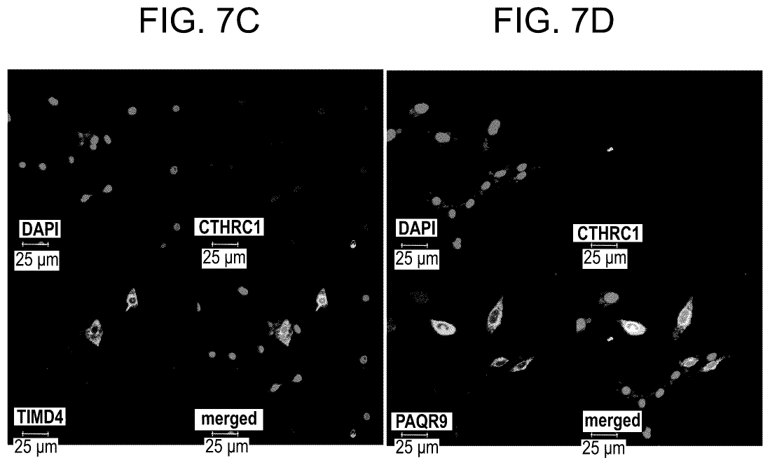

For immunofluorescence staining transfected cells or non-transfected control cells were gently washed with PBS, followed by incubation with 1 ml of VVD-CTHRC1 conditioned media or control conditioned media on ice for 1 hour. Coverslips were washed four times with 2 ml of cold PBS and then incubated with Vli13E09-Alexa 488 (100 ng/ml) for 2 hours at 4° C. The cells on coverslips were washed gently four times with cold PBS, then fixed with 1.5 ml Formalin for 10 minutes before washing with PBS and PBS-Tween20 (0.05%, PBS-T). Non-specific antibody binding to cells was blocked with PBS-T containing 1% bovine serum albumin (BSA) for 30 minutes, followed by overnight incubation at 4° C. with anti-Myc tag antibody clone Vli01 conjugated to Alexa-Fluor647 (100 ng/ml, ThermoFisher #A20006). Cells were washed 4 times with PBS-T before incubating with Hoechst 33342 (1 μg/ml) for 5 minutes. The cells on the coverslips were washed again and mounted on microscope slides for confocal microscopy. The results of these experiments are shown in FIGS. 7A-7D and show that VVD-CTHRC1 only binds to TIMD4 expressing cells and not to cells expressing the PAQR9 receptor. The latter is also a transmembrane cell surface receptor identified in the RNA-seq study as highly enriched in the cell population binding VVD-CTHRC1 (FIGS. 6A-6B). Collectively the results show that TIMD4 specifically binds VVD-CTHRC1.

### Example 9: Exemplary Sequences

TABLE 1

| Antibody | | Amino Acid Sequence | | |
|---|---|---|---|---|
| 13E09 | VH chain | QVQLQQSGPELVRPGASVKMSCKASGYTFTTYWMHWVK | | |
| | | QRPGQGLEWIGMNDPANSEIRLNQKFKDKATLNVDKSSNT | | |
| | | AYMQLNRLTSEDSAVYYCARWIYGSGYALDYWGQGTSVT | | |
| | | VSS | | |
| | | (SEQ ID NO: 1) | | |
| | Heavy chain | AKTTPPSVYPLAPGSAAQTNSMVTLGCLVKGYFPEPVTVT | | |
| | constant | WNSGSLSSGVHTFPAVLQSDLYTLSSSVTVPSSTWPSETVT | | |
| | | CNVAHPASSTKVDKKIVPRDCGCKPCICTVPEVSSVFIFPPK | | |
| | | PKDVLTITLTPKVTCVVVDISKDDPEVQFSWFVDDVEVHTA | | |
| | | QTQPREEQFNSTFRSVSELPIMHQDWLNGKEFKCRVNSAAF | | |
| | | PAPIEKTISKTKGRPKAPQVYTIPPPKEQMAKDKVSLTCMIT | | |
| | | DFFPEDITVEWQWNGQPAENYKNTQPIMDTDGSYFVYSKL | | |
| | | NVQKSNWEAGNTFTCSVLHEGLHNHHTEKSLSHSPGK | | |
| | | (SEQ ID NO: 2) | | |
| | VL chain | DIVLTQSPASLAVSLGQRATISCRASKSVSVSGYSYMHWYQ | | |
| | | QKPGQPPKLLIYLASNLESGVPARFSGSGSGTDFTLSIHPVE | | |
| | | EEDAASYYCQLSRELPYTFGGGTKLEIK | | |
| | | (SEQ ID NO: 3) | | |
| | Light chain | RADAAPTVSIFPPSSEQLTSGGASVVCFLNNFYPKDINVKW | | |
| | constant | KIDGSERQNGVLNSWTDQDSKDSTYSMSSTLTLTKDEYER | | |
| | | HNSYTCEATHKTSTSPIVKSFNRNEC | | |
| | | (SEQ ID NO: 4) | | |
| | CDR | Kabat CDR | IMGT | Chothia CDR |
| | HCDR1 | TYWMH | GYTFTTYW | GYTFTTY |
| | | (SEQ ID NO: 5) | (SEQ ID NO: 11) | (SEQ ID NO: 15) |
| | HCDR2 | MNDPANSEIRLN | NDPANSEI | DPANSE |
| | | QKFKD | (SEQ ID NO: 12) | (SEQ ID NO: 16) |
| | | (SEQ ID NO: 6) | | |
| | HCDR3 | WIYGSGYALDY | ARWIYGSGYALDY | WIYGSGYALDY |
| | | (SEQ ID NO: 7) | (SEQ ID NO: 13) | (SEQ ID NO: 7) |
| | LCDR1 | RASKSVSVSGYS | KSVSVSGYSY | RASKSVSVSGYS |
| | | YMH | (SEQ ID NO: 14) | YMH |
| | | (SEQ ID NO: 8) | | (SEQ ID NO: 8) |
| | LCDR2 | LASNLES | LAS | LASNLES |
| | | (SEQ ID NO: 9) | | (SEQ ID NO: 9) |
| | LCDR3 | QLSRELPYT | QLSRELPYT | QLSRELPYT |
| | | (SEQ ID NO: 10) | (SEQ ID NO: 10) | (SEQ ID NO: 10) |

TABLE 2

| Antibody | | Amino Acid Sequence | | |
|---|---|---|---|---|
| 7H11 | VH chain | QVQLQQPGSELVRPGASVKLSCKASGYTFTNYWIHWVKQ | | |
| | | RHGQGLEWIGSIYPGSGIIKYDEKFKSKGTLTVDTSSNT | | |
| | | AYMYLSSLTSEDSAVYYCTRNADGYYGDYAMDYWGQGTS | | |
| | | VTVSS | | |
| | | (SEQ ID NO: 17) | | |
| | Heavy chain | EFAATMHSSALLCCLVLLTGVRAQVQLQQPGSELVRPGA | | |
| | | SVKLSCKASGYTFTNYWIHWVKQRHGQGLEWIGSIYPGS | | |
| | | GIIKYDEKFKSKGTLTVDTSSNTAYMYLSSLTSEDSAVY | | |
| | | YCTRNADGYYGDYAMDYWGQGTSVTVSS | | |
| | | (SEQ ID NO: 18) | | |
| | Heavy chain | AKTTPPSVYPLAPGSAAQTNSMVTLGCLVKGYFPEPVTV | | |
| | constant | TWNSGSLS | | |
| | fragment | (SEQ ID NO: 47) | | |
| | VL chain | DIVLTQSPAFMSASPGEKVTIICSASSSVSYMHWFQQKP | | |
| | | GTSPRLWIYTTSNLASGVPARFSGSGSGTSYSLTISRME | | |
| | | AEDAATYYCQQRNSYPFTFGSGTKLEIKR | | |
| | | (SEQ ID NO: 19) | | |
| | Light chain | EFAATMHSSALLCCLVLLTGVRADIVLTQSPAFMSASPG | | |
| | | EKVTIICSASSSVSYMHWFQQKPGTSPRLWIYTTSNLAS | | |
| | | GVPARFSGSGSGTSYSLTISRMEAEDAATYYCQQRNSYP | | |
| | | FTFGSGTKLEIK | | |
| | | (SEQ ID NO: 43) | | |
| | | Kabat CDR | IMGT | Chothia CDR |
| | HCDR1 | NYWIH | GYTFTNYW | GYTFTNY |
| | | (SEQ ID NO: 20) | (SEQ ID NO: 44) | (SEQ ID NO: 41) |
| | HCDR2 | SIYPGSGIIKY | IYPGSGII | YPGSGI |
| | | DEKFKS | (SEQ ID NO: 45) | (SEQ ID NO: 42) |
| | | (SEQ ID NO: 21) | | |
| | HCDR3 | NADGYYGDY | TRNADGYYG | NADGYYGDY |
| | | AMDY | DYAMDY | AMDY |
| | | (SEQ ID NO: 22) | (SEQ ID NO: 39) | (SEQ ID NO: 22) |
| | LCDR1 | SASSSVSYMH | SSVSY | SASSSVSYMH |
| | | (SEQ ID NO: 23) | (SEQ ID NO: 40) | (SEQ ID NO: 23) |
| | LCDR2 | TTSNLAS | TT | TTSNLAS |
| | | (SEQ ID NO: 24) | | (SEQ ID NO: 24) |
| | LCDR3 | QQRNSYPFT | QQRNSYPFT | QQRNSYPFT |
| | | (SEQ ID NO: 25) | (SEQ ID NO: 25) | (SEQ ID NO: 25) |

TABLE 3

| Protein | Amino Acid Sequence |
|---|---|
| Signal peptide | MGWSCIILFLAATATGVHS |
| | (SEQ ID NO: 26) |
| | METDTLLLWVLLLWVPGSTG |
| | (SEQ ID NO: 27) |

TABLE 4

| Description | Nucleotide Sequence |
|---|---|
| 13E09 | CAGGTCCAACTGCAGCAATCTGGGCCTGAGCTGGTGAGGCCTGG |
| VH | GGCTTCAGTGAAGATGTCCTGCAAGGCTTCAGGCTATACCTTCA |
| | CCACCTACTGGATGCACTGGGTGAAACAGAGGCCTGGACAAGG |
| | CCTTGAGTGGATTGGCATGAATGATCCTGCGAATAGTGAAATTA |
| | GGTTAAATCAGAAATTCAAGGACAAGGCCACATTGAATGTAGA |
| | CAAATCCTCCAACACAGCCTACATGCAGCTCAACAGACTGACAT |
| | CTGAGGACTCTGCAGTCTATTACTGTGCAAGATGGATCTACGGT |
| | AGTGGCTACGCTTTGGACTACTGGGGTCAAGGAACCTCAGTCAC |
| | CGTCTCCTCA |
| | (SEQ ID NO: 28) |
| 13E09 | GCCAAAACGACACCCCCATCTGTCTATCCACTGGCCCCTGGATC |
| Heavy chain | TGCTGCCCAAACTAACTCCATGGTGACCCTGGGATGCCTGGTCA |
| constant | AGGGCTATTTCCCTGAGCCAGTGACAGTGACCTGGAACTCTGGA |
| | TCCCTGTCCAGCGGTGTGCACACCTTCCCAGCTGTCCTGCAGTCT |
| | GACCTCTACACTCTGAGCAGCTCAGTGACTGTCCCCTCCAGCAC |
| | CTGGCCCAGCGAGACCGTCACCTGCAACGTTGCCCACCCGGCCA |
| | GCAGCACCAAGGTGGACAAGAAAATTGTGCCCAGGGATTGTGG |
| | TTGTAAGCCTTGCATATGTACAGTCCCAGAAGTATCATCTGTCTT |
| | CATCTTCCCCCCAAAGCCCAAGGATGTGCTCACCATTACTCTGA |
| | CTCCTAAGGTCACGTGTGTTGTGGTAGACATCAGCAAGGATGAT |
| | CCCGAGGTCCAGTTCAGCTGGTTTGTAGATGATGTGGAGGTGCA |
| | CACAGCTCAGACGCAACCCCGGGAGGAGCAGTTCAACAGCACT |
| | TTCCGCTCAGTCAGTGAACTTCCCATCATGCACCAGGACTGGCT |
| | CAATGGCAAGGAGTTCAAATGCAGGGTCAACAGTGCAGCTTTCC |
| | CTGCCCCCATCGAGAAAACCATCTCCAAAACCAAAGGCAGACC |
| | GAAGGCTCCACAGGTGTACACCATTCCACCTCCCAAGGAGCAG |
| | ATGGCCAAGGATAAAGTCAGTCTGACCTGCATGATAACAGACTT |
| | CTTCCCTGAAGACATTACTGTGGAGTGGCAGTGGAATGGGCAGC |
| | CAGCGGAGAACTACAAGAACACTCAGCCCATCATGGACACAGA |
| | TGGCTCTTACTTCGTCTACAGCAAGCTCAATGTGCAGAAGAGCA |
| | ACTGGGAGGCAGGAAATACTTTCACCTGCTCTGTGTTACATGAG |
| | GGCCTGCACAACCACCATACTGAGAAGAGCCTCTCCCACTCTCC |
| | TGGTAAA |
| | (SEQ ID NO: 29) |
| 13E09 | GACATTGTGCTGACACAGTCTCCTGCTTCCTTAGCTGTATCTCTG |
| VL | GGGCAGAGGGCCACCATCTCATGCAGGGCCAGCAAAAGTGTCA |
| | GTGTATCTGGCTATAGTTATATGCACTGGTACCAACAGAAACCA |
| | GGACAGCCACCCAAACTCCTCATCTATCTTGCATCCAACCTAGA |
| | ATCTGGGGTCCCTGCCAGGTTCAGTGGCAGTGGGTCTGGGACAG |
| | ACTTCACCCTCAGCATCCATCCTGTGGAGGAGGAGGATGCTGCA |
| | TCCTATTACTGTCAGCTCAGTAGGGAGCTTCCGTACACGTTCGG |
| | CGGGGGGACCAAGCTGGAAATAAAA |
| | (SEQ ID NO: 30) |
| 13E09 | CGGGCTGATGCTGCACCAACTGTATCCATCTTCCCACCATCCAG |
| Light chain | TGAGCAGTTAACATCTGGAGGTGCCTCAGTCGTGTGCTTCTTGA |
| constant | ACAACTTCTACCCCAAAGACATCAATGTCAAGTGGAAGATTGAT |
| | GGCAGTGAACGACAAAATGGCGTCCTGAACAGTTGGACTGATC |
| | AGGACAGCAAAGACAGCACCTACAGCATGAGCAGCACCCTCAC |
| | GTTGACCAAGGACGAGTATGAACGACATAACAGCTATACCTGT |
| | GAGGCCACTCACAAGACATCAACTTCACCCATTGTCAAGAGCTT |
| | CAACAGGAATGAGTGT |
| | (SEQ ID NO: 31) |
| 7H11 | CAGGTCCAACTACAGCAACCTGGGTCTGAGCTGGTGAGGCCTGG |
| VH | AGCTTCAGTGAAGCTGTCCTGCAAGGCTTCTGGCTACACATTCA |
| | CCAACTACTGGATACACTGGGTGAAGCAGAGACATGGACAAGG |
| | CCTTGAGTGGATTGGAAGTATTTATCCTGGTAGTGGTATAATTA |
| | AATACGATGAAAAATTCAAGAGTAAGGGTACATTGACTGTAGA |
| | CACATCCTCCAACACAGCCTACATGTACCTCAGCAGCCTGACAT |
| | CTGAGGACTCTGCGGTCTATTACTGTACAAGAAACGCCGATGGT |
| | TACTATGGTGACTATGCTATGGACTACTGGGGTCAAGGAACCTC |
| | AGTCACCGTCTCCTCA |
| | (SEQ ID NO: 32) |
| 7H11 | GAATTCGCCGCCACCATGCACAGCTCAGCACTGCTCTGTTGCCT |
| Heavy chain | GGTCCTCCTGACTGGGGTGAGGGCCCAGGTGCAGCTGCAGCAG |
| | CCCGGCAGCGAGCTGGTGAGGCCTGGAGCTTCCGTGAAGCTGTC |
| | CTGCAAGGCCTCCGGCTACACATTCACCAACTACTGGATTCACT |
| | GGGTGAAGCAGAGGCACGGCCAGGGCCTGGAGTGGATCGGCTC |
| | CATCTACCCCGGCAGCGGCATCATCAAGTACGATGAGAAGTTTA |
| | AGAGCAAGGGCACCCTGACCGTGGATACAAGCAGCAACACAGC |
| | CTACATGTACCTGAGCTCCCTGACAAGCGAGGACAGCGCCGTGT |
| | ACTACTGCACCAGAAATGCCGATGGCTACTACGGCGACTACGCC |
| | ATGGACTACTGGGGCCAGGGCACCAGCGTGACAGTGTCCTCC |
| | (SEQ ID NO: 33) |
| 7H11 | GCCAAAACGACACCCCCATCTGTCTATCCACTGGCCCCTGGATC |
| Heavy chain | TGCTGCCCAAACTAACTCCATGGTGACCCTGGGATGCCTGGTCA |
| constant | AGGGCTATTTCCCTGAGCCAGTGACAGTGACCTGGAACTCTGGA |
| fragment | TCCCTGTCC (SEQ ID NO: 48) |
| 7H11 | GACATTGTGCTGACACAATCTCCAGCATTCATGTCTGCATCTCC |
| VL | AGGGGAGAAGGTCACCATAATCTGCAGTGCCAGCTCAAGTGTA |
| | AGTTACATGCACTGGTTCCAGCAGAAGCCAGGCACTTCTCCCAG |
| | ACTCTGGATTTATACCACATCCAACCTGGCTTCTGGAGTCCCTGC |
| | TCGCTTCAGTGGCAGTGGATCTGGGACCTCTTACTCTCTCACAAT |
| | CAGCCGAATGGAGGCTGAAGATGCTGCCACTTATTACTGCCAGC |
| | AAAGGAATAGTTACCCATTCACGTTCGGCTCGGGGACAAAGTTG |
| | GAAATAAAACGG |
| | (SEQ ID NO: 34) |
| 7H11 | GAATTCGCCGCCACCATGCACAGCTCAGCACTGCTCTGTTGCCT |
| Light chain | GGTCCTCCTGACTGGGGTGAGGGCCGACATCGTGCTGACACAGA |
| | GCCCTGCCTTTATGTCCGCCAGCCCTGGCGAGAAGGTGACCATC |
| | ATCTGCTCCGCCTCCAGCAGCGTGAGCTACATGCACTGGTTTCA |
| | GCAGAAGCCCGGCACCAGCCCTAGACTGTGGATCTACACCACCT |
| | CCAACCTGGCCTCCGGCGTGCCCGCTAGGTTCTCCGGAAGCGGC |
| | TCCGGCACAAGCTACAGCCTGACCATCAGCAGGATGGAGGCCG |
| | AGGACGCCGCCACATACTACTGTCAGCAGAGAAATTCCTACCCT |
| | TTTACCTTTGGCTCCGGCACCAAGCTGGAGATCAAG |
| | (SEQ ID NO: 46) |

TABLE 5

| Protein | Nucleotide Sequence |
|---|---|
| Signal peptide | ATGGGATGGAGCTGTATCATCCTCTTCTTGG |
| | CAGCAACAGCTACAGGTGTCCACTCC |
| | (SEQ ID NO: 35) |
| | ATGGAGACAGACACACTCCTGTTATGGGTAC |
| | TGCTGCTCTGGGTTCCAGGTTCCACTGGT |
| | (SEQ ID NO: 36) |

### REFERENCES

1. Pyagay, P., et al., Collagen triple helix repeat containing 1, a novel secreted protein in injured and diseased arteries, inhibits collagen expression and promotes cell migration. Circ Res, 2005. 96(2): p. 261-8.
2. Duarte, C. W., et al., Elevated plasma levels of the pituitary hormone Cthrc1 in individuals with red hair but not in patients with solid tumors. PLoS One, 2014. 9(6): p. e100449.
3. Durmus, T., et al., Expression analysis of the novel gene collagen triple helix repeat containing-1 (Cthrc1). Gene Expr Patterns, 2006. 6(8): p. 935-40.
4. LeClair, R. J., et al., Cthrc1 is a novel inhibitor of transforming growth factor-beta signaling and neointimal lesion formation. Circ Res, 2007. 100(6): p. 826-33.
5. LeClair, R. and V. Lindner, The role of collagen triple helix repeat containing 1 in injured arteries, collagen expression, and transforming growth factor beta signaling. Trends Cardiovasc Med, 2007. 17(6): p. 202-5.
6. Leclair, R. J., et al., Intracellular localization of Cthrc1 characterizes differentiated smooth muscle. Arterioscler Thromb Vasc Biol, 2008. 28(7): p. 1332-8.
7. Shekhani, M. T., et al., Collagen triple helix repeat containing 1 is a new promigratory marker of arthritic pannus. Arthritis Res Ther, 2016. 18: p. 171.
8. Liu, G., et al., N-glycosylation induces the CTHRC1 protein and drives oral cancer cell migration. J Biol Chem, 2013. 288(28): p. 20217-27.
9. Ruiz-Villalba, A., et al., Single-Cell RNA Sequencing Analysis Reveals a Crucial Role for CTHRC1 (Collagen Triple Helix Repeat Containing 1) Cardiac Fibroblasts After Myocardial Infarction. Circulation, 2020. 142(19): p. 1831-1847.
10. Nordquist, E. M., et al., Tgfβ1-Cthrc1 Signaling Plays an Important Role in the Short-Term Reparative Response to Heart Valve Endothelial Injury. Arterioscler Thromb Vasc Biol, 2021. 41(12): p. 2923-2942.
11. Apra, C., et al., Cthrc1 is a negative regulator of myelination in Schwann cells. Glia, 2012. 60(3): p. 393-403.
12. Toomey, B. H., et al., Activated CTHRC1 promotes glycolysis in endothelial cells: Implications for metabolism and angiogenesis. Vascul Pharmacol, 2023. 153: p. 107246.
13. Stohn, J. P., et al., Cthrc1 controls adipose tissue formation, body composition, and physical activity, in Obesity. 2015. p. 1633-1642.
14. Gordon, J. A. R., et al., Identification of molecularly unique tumor-associated mesenchymal stromal cells in breast cancer patients, in PLOS ONE. 2023, PLOS. p. e0282473.
15. Jin Y.-R., et al., Inhibition of osteoclast differentiation and collagen antibody-induced arthritis by CTHRC1. Bone 2017; 97:153-167.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

1. An isolated antibody, or antigen-binding fragment thereof, which comprises:
1) a) a heavy chain variable region (VH) comprising an HCDR1 amino acid sequence of SEQ ID NO: 5; an HCDR2 amino acid sequence of SEQ ID NO: 6; and an HCDR3 amino acid sequence of SEQ ID NO: 7; and a light chain variable region (VL) comprising an LCDR1 amino acid sequence of SEQ ID NO: 8, an LCDR2 amino acid sequence of SEQ ID NO: 9, and an LCDR3 amino acid sequence of SEQ ID NO: 10; or
b) VH comprising an HCDR1 amino acid sequence of SEQ ID NO: 20; an HCDR2 amino acid sequence of SEQ ID NO: 21; and an HCDR3 amino acid sequence of SEQ ID NO: 22; and VL comprising an LCDR1 amino acid sequence of SEQ ID NO: 23, an LCDR2 amino acid sequence of SEQ ID NO: 24, and an LCDR3 amino acid sequence of SEQ ID NO: 25,
wherein the HCDR1, HCDR2, HCDR3, LCDR1, LCDR2, and LCDR3 are according to Kabat numbering; or
2) a) a heavy chain variable region (VH) comprising an HCDR1 amino acid sequence of SEQ ID NO: 11; an HCDR2 amino acid sequence of SEQ ID NO: 12; and an HCDR3 amino acid sequence of SEQ ID NO: 13; and a light chain variable region (VL) comprising an LCDR1 amino acid sequence of SEQ ID NO: 14, an LCDR2 amino acid sequence of “LAS”, and an LCDR3 amino acid sequence of SEQ ID NO: 10; or
b) VH comprising an HCDR1 amino acid sequence of SEQ ID NO: 37; an HCDR2 amino acid sequence of SEQ ID NO: 38; and an HCDR3 amino acid sequence of SEQ ID NO: 39; and VL comprising an LCDR1 amino acid sequence of SEQ ID NO: 40, an LCDR2 amino acid sequence of “TT”, and an LCDR3 amino acid sequence of SEQ ID NO: 25,
wherein the HCDR1, HCDR2, HCDR3, LCDR1, LCDR2, and LCDR3 are according to IMGT numbering; or
3) a) a heavy chain variable region (VH) comprising an HCDR1 amino acid sequence of SEQ ID NO: 15; an HCDR2 amino acid sequence of SEQ ID NO: 16; and an HCDR3 amino acid sequence of SEQ ID NO: 7; and a light chain variable region (VL) comprising an LCDR1 amino acid sequence of SEQ ID NO: 8, an LCDR2 amino acid sequence of SEQ ID NO: 9, and an LCDR3 amino acid sequence of SEQ ID NO: 10; or
b) VH comprising an HCDR1 amino acid sequence of SEQ ID NO: 41; an HCDR2 amino acid sequence of SEQ ID NO: 42; and an HCDR3 amino acid sequence of SEQ ID NO: 22; and VL comprising an LCDR1 amino acid sequence of SEQ ID NO: 23, an LCDR2 amino acid sequence of SEQ ID NO: 24, and an LCDR3 amino acid sequence of SEQ ID NO: 25,
wherein the HCDR1, HCDR2, HCDR3, LCDR1, LCDR2, and LCDR3 are according to Chothia numbering.

2.-3. (canceled)

4. The isolated antibody, or antigen-binding fragment thereof of claim 3, wherein the VH comprises an amino acid sequence at least 85%, 90%, or 95% identity to SEQ ID NO: 1 or 17; or
wherein the VL comprises an amino acid sequence at least 85%, 90%, or 95% identity to SEQ ID NO: 3 or 19.

5.-12. (canceled)

13. The isolated antibody, or antigen-binding fragment thereof of claim 1, wherein the VH comprises an amino acid sequence set forth in SEQ ID NO: 1 and the VL comprises an amino acid sequence set forth in SEQ ID NO: 3; or
wherein the VH comprises an amino acid sequence set forth in SEQ ID NO: 17 and the VL comprises an amino acid sequence set forth in SEQ ID NO: 19.

14. (canceled)

15. The isolated antibody, or antigen-binding fragment thereof of claim 1, wherein:
(a) the isolated antibody, or antigen-binding fragment thereof comprises an antigen-binding fragment;
(b) the heavy chain constant region is chosen from the heavy chain constant regions of IgG1, IgG2, IgG3, or IgG4, or a chimera of two or more isotypes (e.g. IgG2/4);
(c) the light chain constant region is chosen from the light chain constant regions of kappa or lambda;
(d) the heavy chain constant region is chosen from the heavy chain constant regions of IgG1, IgG2, IgG3, IgG4, or a chimera of two or more isotypes (e.g. IgG2 and IgG4), and the light chain constant region is chosen from the light chain constant regions of kappa or lambda; and/or
(e) the isolated antibody, or antigen-binding fragment thereof is a humanized antibody, monoclonal antibody, synthetic antibody, monospecific antibody, multispecific antibody, and/or bispecific antibody.

16.-22. (canceled)

23. The isolated antibody, or antigen-binding fragment thereof of claim 1, wherein the isolated antibody, or antigen-binding fragment thereof specifically binds to Collagen Triple Helix Repeat Containing 1 (CTHRC1) or a fragment thereof.

24.-26. (canceled)

27. A pharmaceutical composition comprising the isolated antibody, or antigen-binding fragment thereof of any of claim 1 and a pharmaceutically acceptable carrier, excipient or stabilizer.

28. An isolated nucleic acid encoding the VH and VL of the isolated antibody, or antigen-binding fragment thereof of claim 1.

29. (canceled)

30. A host cell comprising the nucleic acid of claim 28.

31. A method of producing an isolated antibody, or antigen-binding fragment thereof, comprising culturing the host cell of claim 30 under conditions suitable for gene expression.

32. A method of binding or interacting with CTHRC1 or a fragment thereof, comprising contacting CTHRC1 with an isolated antibody, or antigen-binding fragment thereof of claim 1.

33. A method of treating a disease or disorder, comprising administering to a subject in need thereof an isolated antibody, or antigen-binding fragment thereof of claim 1, in an amount effective to treat the disease.

34. The method of claim 33, wherein the disorder or disease is a cancer, optionally, wherein the cancer is chosen from the group comprising of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer; or
wherein the disease or disorder is pre-cancerous cells in the subject.

35.-37. (canceled)

38. The method of claim 33, further comprising administering a second therapeutic agent or modality.

39. (canceled)

40. A method of preventing a disease or disorder, comprising administering to a subject in need thereof an isolated antibody, or antigen-binding fragment thereof of claim 1, in an amount effective to prevent the disorder or disease.

41. The method of claim 40, wherein the disease is a cancer, optionally, wherein the cancer is chosen from the group comprising of melanoma, central nervous system (CNS) cancer, CNS germ cell tumor, lung cancer, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, liver cancer, oral cancer, cervical cancer, kidney cancer, or hematological cancer; or
wherein the disease or disorder is pre-cancerous cells in the subject.

42. (canceled)

43. A method of detecting CTHRC1, comprising (i) contacting a sample or a subject with an isolated antibody, or antigen-binding fragment thereof of claim 1 under conditions that allow interaction of the isolated antibody, or antigen-binding fragment thereof and CTHRC1 to occur, and (ii) detecting formation of a complex between the isolated antibody, or antigen-binding fragment thereof and the sample or subject.

44. An antibody-drug conjugate (ADC) comprising the antibody, or antigen-binding fragment thereof of claim 1 conjugated to at least one drug,
wherein the antibody, or antigen-binding fragment thereof binds collagen triple helix repeat containing 1 (CTHRC1).

45. The ADC of claim 44, wherein the at least one drug is selected from the group consisting of an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a DNA damaging agent, a corticosteroid, a photoactive therapeutic agent, an oligonucleotide, a radionuclide agent, a radiosensitizer, a topoisomerase inhibitor, and a tyrosine kinase inhibitor; and
wherein the at least one drug is conjugated to the antibody, or antigen-binding portion thereof, via a linker.

46.-49. (canceled)

50. A pharmaceutical composition comprising the ADC of claim 44, and a pharmaceutically acceptable carrier.

51. A method of treating or preventing a disease or disorder, comprising administering to a subject a therapeutically effective amount of the ADC of any one of claim 44, thereby treating or preventing the disease or disorder.

52.-53. (canceled)
